(19) United States
(12) Patent Application Publication
(10) Pub. No.: US 2007/0015823 A1
(43) Pub. Date: Jan. 18, 2007

(54) Ambruticin VS compounds

(76) Inventors: Zong-Qiang Tian, Fremont, CA (US); David C. Myles, Kensington, CA (US); Zhan Wang, El Dorado Hills, CA (US); Yuan Xu, Fremont, CA (US); Bryan Julien, Oakland, CA (US); Eduardo Rodriguez, Rosario (AR); Mark Shimazu, Hayward, CA (US); Greg O. Buchanan, Hayward, CA (US); Leonard Katz, Oakland, CA (US)

Correspondence Address: KOSAN BIOSCIENCES, INC, 3832 BAY CENTER PLACE, HAYWARD, CA, 94588, US

(21) Appl. No.: 11/305,802

(22) Filed: Dec. 16, 2005

Related U.S. Application Data
(60) Provisional application No. 60/637,110, filed on Dec. 16, 2004.
(60) Provisional application No. 60/676,446, filed on Apr. 28, 2005.
(60) Provisional application No. 60/683,283, filed on May 19, 2005.

(51) Int. Cl. A61K 31/35; C07D 405/14; C07D 405/02

(52) U.S. Cl. 514/460; 549/415

(57) ABSTRACT

Antifungal compounds represented by formula I 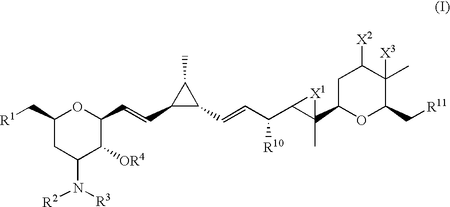
where R¹, R², R³ and R⁴ are as defined herein. Isolated or purified naturally occurring 20,21-dihydro ambruticin compounds. The invention also provides Sorangium cellulosum ambM, ambO, ambP, and ambS mutant strains; a recombinant Sorangium cellulosum strain that produces 24-norambruticin compounds; and a method for treating a fungal infection using a compound of this invention.

### CROSS-REFERENCE TO RELATED APPLICATIONS

This application claims benefit under 35 U.S.C. § 119 to U.S. Provisional Application Ser. Nos. 60/637,110, filed Dec. 16, 2004, 60/676,446, filed Apr. 28, 2005, and 60/683,283, filed May 19, 2005, the entire contents of which are each incorporated herein by reference.

### BACKGROUND OF THE INVENTION

1. Field of the Invention

This invention relates to compounds having anti-fungal activity, methods for their preparation, and methods for their use.

2. Description of Related Art

Ambruticin S (also referred to as Acid S, W 7783, (5S,6R)-5,6-dihydroxypoly-angioic acid, or, sometimes, simply as ambruticin) is an antifungal compound isolated from cultures of Polyangium cellulosum var. fulvum and has the structure shown below. See Strandtmann et al., U.S. Pat. No. 3,804,948 (1974); Barnes et al., Tetrahedron Letters 22 (18), 1751-1754 (1981); Kende et al., J. Am. Chem. Soc. 112 (26), 9645-9646 (1990).

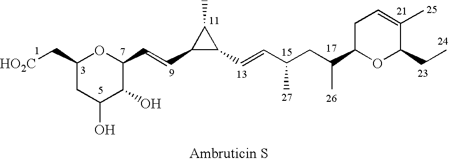

Subsequently, another research group isolated from cultures of Sorangium cellulosum strain Se ce10 a series of six structurally closely related compounds having at C5 an amino group instead of a hydroxyl group. Bedorf et al., WO 91/00860 (1991); Höfle et al., Liebigs Ann. Chem. 1991, 941-945. These compounds have been named ambruticin VS-1, VS-2, VS-3 (or (5S,6R)-5-(dimethylamino)-6-hydroxypolyangioic acid), and so on, and have the structures shown below. (Herein, ambruticin S and the VS-series compounds are collectively referred to as “the ambruticins” and ambruticin compounds other than the aforementioned naturally occurring ones having an oxygen at position C5 will be identified by an “S” designation while those having a nitrogen at position C5 will be identified by a “VS” designation.

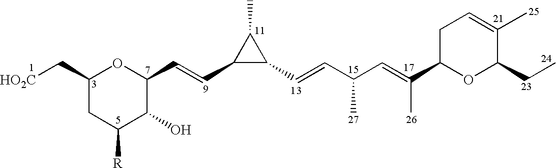

| | |
|---|---|
| VS-1: | R = NMe₃⁺ |
| VS-2: | Me ester of VS-1 |
| VS-3: | R = NMe₂ |
| VS-4: | R = NHMe |
| VS-5: | R = NH₂ |
| VS-3-N-oxide: | R = N⁺(Me₂)O⁻ |

Other disclosures relating to the chemistry or mechanism of action of the ambruticins include: Connor et al., U.S. Pat. No. 3,932,620 (1976); Connor et al., U.S. Pat. No. 3,932,621 (1976); Connor et al., U.S. Pat. No. 4,001,398 (1977); Connor et al., U.S. Pat. No. 4,009,261 (1977); Connor et al., U.S. Pat. No. 4,016,257 (1977); Connor et al., U.S. Pat. No. 4,098,998 (1978); Connor et al., U.S. Pat. No. 4,107,429 (1978); Connor et al., U.S. Pat. No. 4,138,550 (1979); Connor et al., U.S. Pat. No. 4,191,825 (1979); Connor et al., U.S. RE 30,339 (1980); Connor et al., DE 2,659,575 (1978) (Chem. Abs. 89:109030); Connor et al., J. Med. Chem. 22 (9), 1055-1059 (1979); Connor et al., J. Med. Chem. 22 (9), 1144-1147 (1979); and Knauth et al., J. Antibiotics 53 (10), 1182-1190 (2000). The disclosures of the foregoing documents and the other documents cited in this BACKGROUND OF THE INVENTION section are incorporated herein by reference.

### BRIEF SUMMARY OF THE INVENTION

In a first aspect of the invention, there is provided a compound represented by formula I 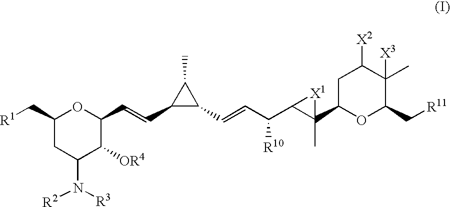
and the pharmaceutically acceptable salts, solvates, hydrates, and prodrug forms thereof, wherein
R¹⁰ and R¹¹ are independently H or CH₃;
X¹ is either a bond or O;
X² and X³ are each H or together are a bond;
R¹ is 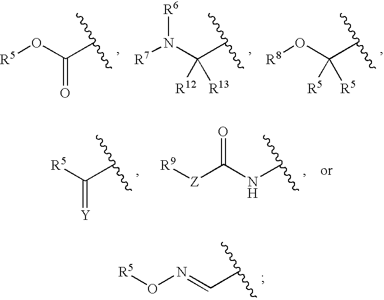
R² and R³ are independently H, C₁-C₅ alkyl, C₂-C₅ alkenyl, C₂-C₅ alkynyl, cycloalkyl, aryl(C₁-C₅ alkyl), aryl(C₂-C₅ alkenyl), aryl(C₂-C₅ alkynyl), cycloalkyl(C₁-C₅ alkyl), cycloalkyl(C₂-C₅ alkenyl), cycloalkyl(C₂-C₅ alkynyl), 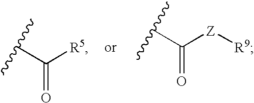
R⁴ is H, 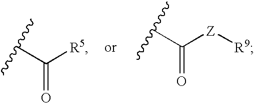
   or R³ and R⁴ combine to form 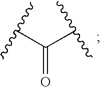
R⁵ is, independently for each occurrence thereof, H, C₁-C₅ alkyl, C₂-C₅ alkenyl, C₂-C₅ alkynyl, cycloalkyl, or aryl;
R⁶ and R⁷ are independently H, C₁-C₅ alkyl, C₂-C₅ alkenyl, C₂-C₅ alkynyl, cycloalkyl, or aryl; or R⁶ and R⁷ and the nitrogen to which they are commonly bonded combine to form an aziridinyl, azetidinyl, pyrrolidinyl, or piperidinyl ring;
R⁸ is R⁵ or 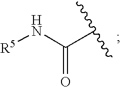
R⁹ is, independently for each occurrence thereof, H, C₁-C₅ alkyl, C₂-C₅ alkenyl, C₂-C₅ alkynyl, cycloalkyl, aryl(C₁-C₅ alkyl), aryl(C₂-C₅ alkenyl), aryl(C₂-C₅ alkynyl), cycloalkyl(C₁-C₅ alkyl), cycloalkyl(C₂-C₅ alkenyl), or cycloalkyl(C₂-C₅ alkynyl), provided that R⁹ is not H when Z is O;
R¹² and R¹³ together are 0, or R¹² is H and R¹³ is R⁵;
Y is O or N—OR⁵; and
Z is, independently for each occurrence thereof, O or NH;
with the proviso that when R¹ is 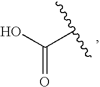
   R¹⁰ and R¹¹ are both CH₃, X¹ is a bond, and R² is H or CH₃, then R³ is other than H or CH₃.

In a second aspect, there is provided a method of treating or reducing the probability of a fungal infection in a subject in need of such treatment, comprising administering a pharmaceutical composition comprising a therapeutically effective amount of a compound of this invention and optionally a pharmaceutically acceptable carrier.

In a third aspect, there is provided the use of a compound of this invention for the preparation of a medicament for treating a fungal infection.

In a fourth aspect, there is provided a pharmaceutical formulation comprising a compound of this invention and an excipient.

In a fifth aspect, there is provided an isolated or recombinant cell comprising the genes of the ambruticin biosynthetic gene cluster and producing one or more ambruticins or ambruticin analogs, wherein the activity the ambP, ambO, ambS, or ambM gene product is reduced or disrupted. The isolated or recombinant cell, wherein the activity the ambM gene product is reduced or disrupted, produces an ambruticin analog in which R¹⁰ is H. The isolated or recombinant cell, wherein the activity the ambP and/or ambO gene product(s) is/are reduced or disrupted, produces an ambruticin analog in which X² and X³ are each H. The isolated or recombinant cell, wherein the activity the ambS gene product is reduced or disrupted, produces elevated amounts of ambruticin VS-5 and ambruticin S and does not produce ambruticin VS-1, ambruticin VS-2, ambruticin VS-3 and ambruticin VS-4.

In a sixth aspect, there is provided an isolated or recombinant cell comprising the genes of the ambruticin biosynthetic gene cluster, wherein the malonate specific AT domain from module 7 is replaced or engineered into a loading domain. The isolated or recombinant cell produces an ambruticin analog in which R¹¹ is H.

In a seventh aspect, there is provided a method of producing one or more ambruticins or ambruticin analogs comprising culturing the isolated or recombinant cell comprising the genes of the ambruticin biosynthetic gene cluster and producing one or more ambruticins or ambruticin analogs, wherein the activity the ambP, ambO, ambS, or ambM gene product is reduced or disrupted, or the malonate specific AT domain from module 7 is replaced or engineered into a loading domain, or a combination thereof.

In an eighth aspect, there is provided an isolated or purified compound represented by the formula (II-D): 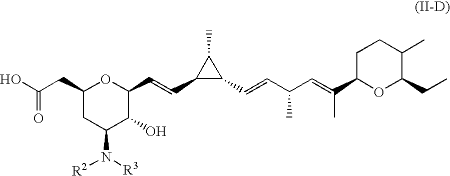
wherein R² and R³ are, independently for each occurrence thereof, H or CH₃.

### BRIEF DESCRIPTION OF THE DRAWINGS

FIG. 1 shows the HPLC chromatograms of extracts from cultures of the Sorangium cellulosum So ce10 wild-type strain (Panel A) and the ambS⁻ (Panel B) and ambP⁻ (Panel C) mutants. The ambP⁻ mutant gave a chromatogram identical to that of the ambP⁻ mutant. Strains were grown under identical conditions and analyzed by the VS method described in Example 22. Peaks representing compounds for which a purified standard was available are indicated.

FIG. 2 shows the HPLC chromatograms of the Sorangium cellulosum So ce10 wild-type strain (Panel A) and the ambM⁻ mutant (Panel B). Strains were grown under identical conditions and analyzed by the VS method described in Example 22. Peaks representing compounds for which a purified standard was available are indicated.

### DETAILED DESCRIPTION OF THE INVENTION

Definitions

“Alkyl” means an optionally substituted straight or branched chain hydrocarbon moiety having the specified number of carbon atoms in its longest chain portion (e.g., as in “C₃ alkyl,” “C₁-C₅ alkyl,” or “C₁ to C₅ alkyl,” the latter two phrases referring to an alkyl group having from 1 to 5 carbon atoms in the longest chain portion) or, where the number of carbon atoms is not specified, from 1 to 4 carbon atoms in the longest chain portion.

“Alkenyl” means an optionally substituted straight or branched chain hydrocarbon moiety having at least one carbon-carbon double bond and the specified number of carbon atoms in its longest chain portion (e.g., as in “C₃ alkenyl,” “C₂-C₅ alkenyl,” or “C₂ to C₅ alkenyl,” the latter two phrases referring to an alkenyl group having from 2 to 5 carbon atoms in the longest chain portion) or, where the number of carbon atoms is not specified, from 2 to 4 carbon atoms in the longest chain portion.

“Alkynyl” means an optionally substituted straight or branched chain hydrocarbon moiety having at least one carbon-carbon triple bond and the specified number of carbon atoms in its longest chain portion (e.g., as in “C₃ alkenyl,” “C₂-C₅ alkynyl,” or “C₂ to C₅ alkynyl,” the latter two phrases referring to an alkynyl group having from 2 to 5 carbon atoms in the longest chain portion) or, where the number of carbon atoms is not specified, from 2 to 4 carbon atoms in the longest chain portion.

“Aryl” means an aromatic monocyclic, fused bicyclic, or fused polycyclic hydrocarbon or heterocyclic group having 1 to 20 carbon atoms in the ring portion(s), such as phenyl, napthyl, pyrrolyl, indolyl, pyrazolyl, pyrazolinyl, imidazolyl, oxazolyl, isoxazolyl, thiazolyl, thiadazolyl, isothiazolyl, furyl, thienyl, oxadiazolyl, pyridinyl, N-oxo-pyridyl, pyrazinyl, pyrimidinyl, pyridazinyl, tetrazinyl, triazinyl, triazolyl, benzothiazolyl, benzoxazolyl, benzothienyl, quinolinyl, quinolinyl-N-oxide, isoquinolinyl, benzimidazolyl, benzofuryl, chromonyl, coumarinyl, cinnolinyl, quinoxalinyl, indazolyl, benzisothiazolyl, benzisoxazolyl, benzodiazinyl, tetrazolyl, benzofurazanyl, benzothiopyranyl, benzpyrazolyl, indolinyl, isochromanyl, isoindolinyl, naphthyridinyl, phthalazinyl, purinyl, quinazolinyl, and the like. Aryl groups may be optionally substituted.

“Arylalkyl,”(cycloalkyl)alkyl,” “arylalkenyl,” “arylalkynyl,” “biarylalkyl,” and the like mean an aryl, cycloalkyl, or biaryl group, as the case may be, bonded directly to an alkyl, alkenyl, or alkynyl moiety, as the case may be, with the open (unsatisfied) valence at the alkyl, alkenyl, or alkynyl group, for example as in benzyl, phenethyl, N-imidazoylethyl, N-morpholinoethyl, and the like.

“Cycloalkyl” means an optionally substituted, saturated or unsaturated, non-aromatic cyclic hydrocarbon ring system, preferably containing 1 to 3 rings and 3 to 7 carbons per ring which may be further fused with a saturated or unsaturated C₃-C₇ carbocyclic ring. Exemplary cycloalkyl ring systems include cyclopropyl, cyclobutyl, cyclopentyl, cyclohexyl, cycloheptyl, cyclooctyl, cyclodecyl, cyclododecyl, and adamantyl, especially the first four listed.

“Halogen” or “halo” means fluorine, chlorine, bromine or iodine.

Pharmaceutically acceptable ester” means an ester that hydrolyzes in vivo (for example in the human body) to produce the parent compound or a salt thereof or has per se activity similar to that of the parent compound. Suitable ester groups include, without limitation, those derived from pharmaceutically acceptable aliphatic carboxylic acids, particularly alkanoic, alkenoic, cycloalkanoic and alkanedioic acids, in which each alkyl or alkenyl moiety preferably has no more than six carbon atoms. Illustrative esters include formates, acetates, propionates, butyrates, acrylates, citrates, succinates, and ethylsuccinates.

“Pharmaceutically acceptable salt” means a salt of a compound suitable for pharmaceutical formulation. Where a compound has one or more basic functionalities, the salt can be an acid addition salt, such as a sulfate, hydrobromide, tartrate, mesylate, maleate, citrate, phosphate, acetate, pamoate (embonate), hydroiodide, nitrate, hydrochloride, lactate, methylsulfate, fumarate, benzoate, succinate, mesylate, lactobionate, suberate, tosylate, and the like. Where a compound has one or more acidic moieties, the salt can be a salt such as a calcium salt, potassium salt, magnesium salt, meglumine salt, ammonium salt, zinc salt, piperazine salt, tromethamine salt, lithium salt, choline salt, diethylamine salt, 4-phenyl-cyclohexylamine salt, benzathine salt, sodium salt, tetramethylammonium salt, and the like.

Where it is indicated that a group may be substituted, for example by use of “substituted or unsubstituted” or “optionally substituted” phrasing, such group may have one or more independently selected substituents, preferably one to five in number, more preferably one or two in number. It is understood that substituents and substitution patterns can be selected by one of ordinary skill in the art to provide compounds that are chemically stable and that can be synthesized by techniques known in the art as well as the methods set forth herein. Examples of suitable substituents include alkyl, alkenyl, alkynyl, aryl, halo, trifluoromethoxy, trifluoromethyl, hydroxy, alkoxy, cycloalkyloxy, heterocyclooxy, alkanoyl, alkanoyloxy, amino, alkylamino quarternary ammonium, aralkylamino, cycloalkylamino, heterocycloamino, dialkylamino, alkanoylamino, thio, alkylthio, cycloalkylthio, heterocyclothio, ureido, nitro, cyano, carboxy, caroboxylalkyl, carbamyl, alkoxycarbonyl, alkylthiono, arylthiono, alkylsulfonyl, sulfonamindo, aryloxy, and the like, in addition to those specified herein. Preferably, the substituent(s) for alkyl, alkenyl, and alkynyl moieties are from one to three in number and are independently selected from N-pyrrolidinyl, N-morpholinyl, N-azetidinyl, hydroxyl, halo, alkoxyl, cyano, amino, alkylamino, and dialkylamino, especially hydroxyl, halo, amino, and alkoxyl. Preferably, the substituent(s) for aryl, cycloalkyl, and heterocycloalkyl moieties are from one to three in number and are independently selected from alkyl, alkenyl, alkynyl, hydroxyalkyl, haloalkyl, hydroxyl, halo, alkoxyl, cyano, aminoalkyl, alkylaminoalkyl, dialkylaminoalkyl, amino, alkylamino, and dialkylamino.

“Therapeutically effective amount” means that amount of active compound(s) or pharmaceutical agent(s) that elicit the biological or medicinal response in a tissue system, animal or human sought by a researcher, veterinarian, medical doctor or other clinician, which response includes alleviation of the symptoms of the disease or disorder being treated. The specific amount of active compound(s) or pharmaceutical agent(s) needed to elicit the biological or medicinal response will depend on a number of factors, including but not limited to the disease or disorder being treated, the active compound(s) or pharmaceutical agent(s) being administered, the method of administration, and the condition of the patient.

Where a range is stated, as in “C₁-C₅ alkyl” or “5 to 10%,” such range includes the end points of the range.

Compounds and Methods

Preferably, in formula I, the stereochemistry at C5 is S.

In a preferred embodiment, R¹ is 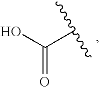
R⁴ is H, and C5 has S stereochemistry, corresponding to a compound represented by formula II-A 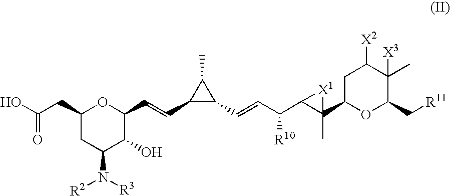
where R², R³, R¹⁰, R¹¹, X¹, X² and X³ are as defined in the BRIEF SUMMARY OF THE INVENTION section hereinabove.

In another preferred embodiment, R¹ is 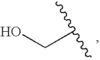
R⁴ is H, and C5 has S stereochemistry, corresponding to a compound represented by formula III: 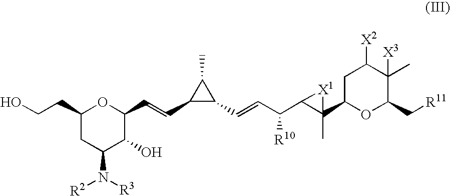
where R², R³, R¹⁰, R¹¹, X¹, X² and X³ are as defined in the BRIEF SUMMARY OF THE INVENTION section hereinabove. In a preferred embodiment of compounds of formula III, R² and R³ are each CH₃.

In another preferred embodiment, R¹ is 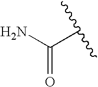
and C5 has S stereochemistry, corresponding to a compound represented by formula IV 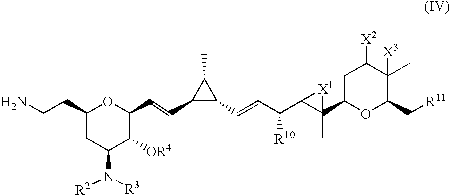
where R², R³, R⁴, R¹⁰, R¹¹, X¹, X² and X³ are as defined in the BRIEF SUMMARY OF THE INVENTION section hereinabove.

In one embodiment, in formula I, R¹⁰ and R¹¹ are each CH₃, X¹ is a bond, and X² and X³ together are a bond, corresponding to a compound represented by formula I-A

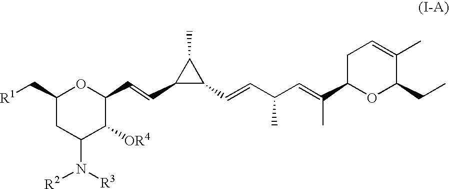

Preferably, in formula I-A, the stereochemistry at C5 is S.

In a preferred embodiment, in formula I-A, R¹ is 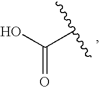
R⁴ is H, and C5 has S stereochemistry, corresponding to a compound represented by formula II-A 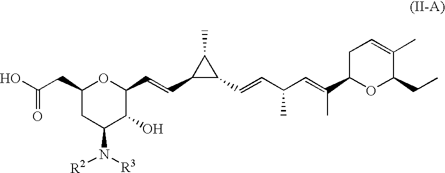
where R² and R³ are as defined in the BRIEF SUMMARY OF THE INVENTION section hereinabove.

In another preferred embodiment, in formula I-A, R¹ is 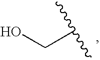
R⁴ is H, and C5 has S stereochemistry, corresponding to a compound represented by formula III-A: 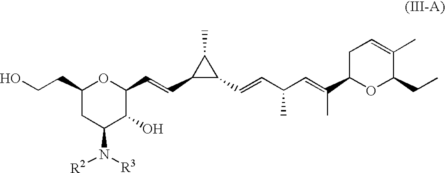
where R² and R³ are as defined in the BRIEF SUMMARY OF THE INVENTION section hereinabove. In a preferred embodiment of compounds of formula III-A, R² and R³ are each CH₃.

In another preferred embodiment, in formula I-A, R¹ is 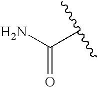
and C5 has S stereochemistry, corresponding to a compound represented by formula IV-A 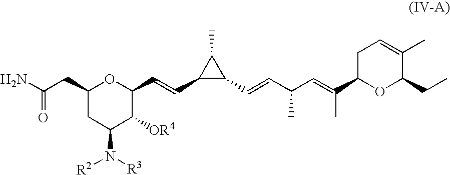
where R², R³ and R⁴ are as defined in the BRIEF SUMMARY OF THE INVENTION section hereinabove.

In one embodiment, in formula I, R¹⁰ is H, R¹¹ is CH₃, X¹ is a bond, and X² and X³ together are a bond, corresponding to a compound represented by formula I-B

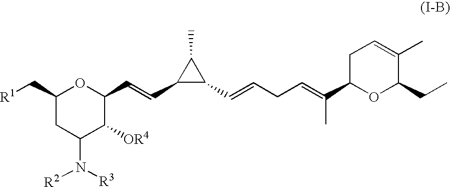

Preferably, in formula I-B, the stereochemistry at C5 is S.

In a preferred embodiment, in formula I-B, R¹ is 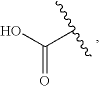
R⁴ is H, and C5 has S stereochemistry, corresponding to a compound represented by formula II-B 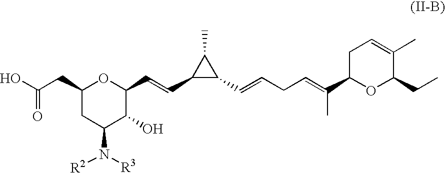
where R² and R³ are as defined in the BRIEF SUMMARY OF THE INVENTION section hereinabove.

In another preferred embodiment, in formula I-B, R¹ is 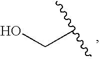
R⁴ is H, and C5 has S stereochemistry, corresponding to a compound represented by formula III-B: 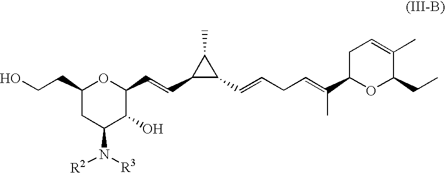
where R² and R³ are as defined in the BRIEF SUMMARY OF THE INVENTION section hereinabove. In a preferred embodiment of compounds of formula II-B, R² and R³ are each CH₃.

In another preferred embodiment, in formula I-B, R¹ is 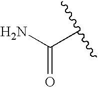
and C5 has S stereochemistry, corresponding to a compound represented by formula IV-B 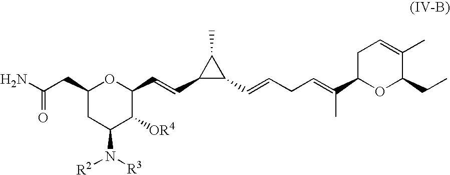
where R², R³ and R⁴ are as defined in the BRIEF SUMMARY OF THE INVENTION section hereinabove.

In one embodiment, in formula I, R¹⁰ is CH₃, R¹¹ is H, X¹ is a bond, and X² and X³ together are a bond, corresponding to a compound represented by formula I-C

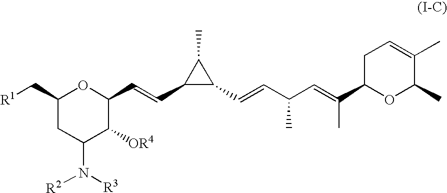

Preferably, in formula I-C, the stereochemistry at C5 is S.

In a preferred embodiment, in formula I-C, R¹ is 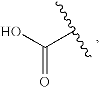
R⁴ is H, and C5 has S stereochemistry, corresponding to a compound represented by formula II-C 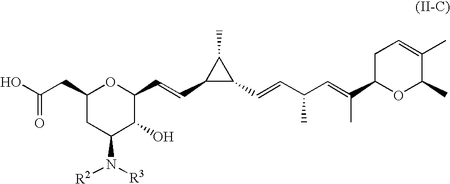
where R² and R³ are as defined in the BRIEF SUMMARY OF THE INVENTION section hereinabove.

In another preferred embodiment, in formula I-C, R¹ is 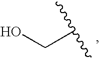
R⁴ is H, and C5 has S stereochemistry, corresponding to a compound represented by formula III-C: 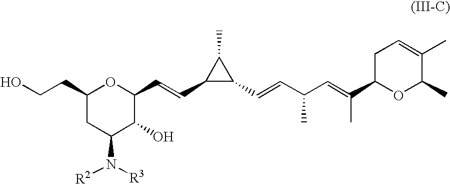
where R² and R³ are as defined in the BRIEF SUMMARY OF THE INVENTION section hereinabove. In a preferred embodiment of compounds of formula III-C, R² and R³ are each CH₃.

In another preferred embodiment, in formula I-C, R¹ is 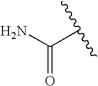
and C5 has S stereochemistry, corresponding to a compound represented by formula IV-C 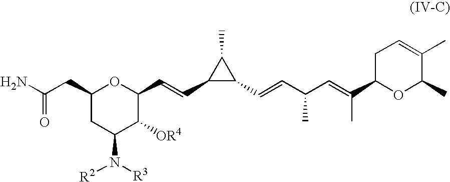
where R², R³ and R⁴ are as defined in the BRIEF SUMMARY OF THE INVENTION section hereinabove.

In one embodiment, in formula I, R¹⁰ and R¹¹ are both CH₃, X¹ is a bond, and X² and X³ are each H, corresponding to a compound represented by formula I-D

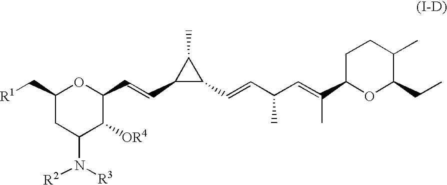

Preferably, in formula I-D, the stereochemistry at C5 is S.

In a preferred embodiment, in formula I-D, R¹ is 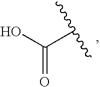
R⁴ is H, and C5 has S stereochemistry, corresponding to a compound represented by formula II-D 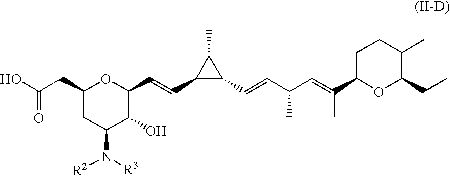
where R² and R³ are as defined in the BRIEF SUMMARY OF THE INVENTION section hereinabove.

In another preferred embodiment, in formula I-D, R¹ is 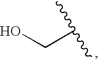
R⁴ is H, and C5 has S stereochemistry, corresponding to a compound represented by formula III-D: 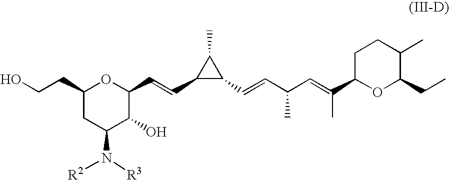
where R² and R³ are as defined in the BRIEF SUMMARY OF THE INVENTION section hereinabove. In a preferred embodiment of compounds of formula III-D, R² and R³ are each CH₃.

In another preferred embodiment, in formula I-D, R¹ is 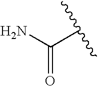
and C5 has S stereochemistry, corresponding to a compound represented by formula IV-D

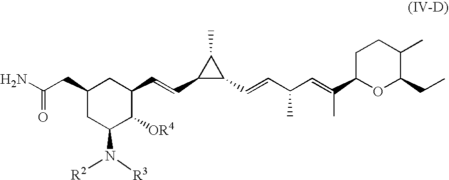

where R², R³ and R⁴ are as defined in the BRIEF SUMMARY OF THE INVENTION section hereinabove.

In one embodiment, in formula I, R¹⁰ and R¹¹ are both CH₃, X¹ is O, and X² and X³ together are a bond, corresponding to a compound represented by formula I-E

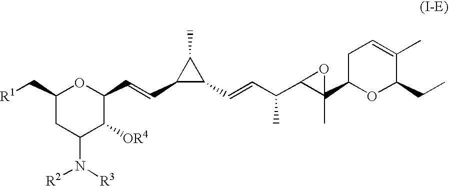

Preferably, in formula I-E, the stereochemistry at C5 is S.

In a preferred embodiment, in formula I-E, R¹ is 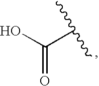
R⁴ is H, and C5 has S stereochemistry, corresponding to a compound represented by formula II-E 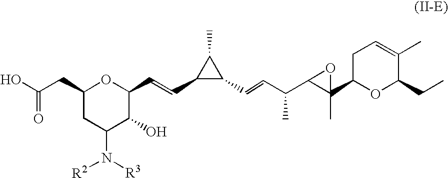
where R² and R³ are as defined in the BRIEF SUMMARY OF THE INVENTION section hereinabove.

In another preferred embodiment, in formula I-E, R¹ is 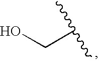
R⁴ is H, and C5 has S stereochemistry, corresponding to a compound represented by formula III-E: 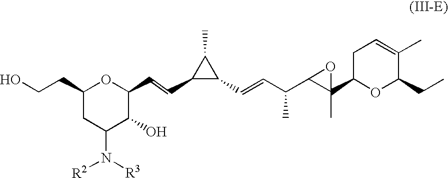
where R² and R³ are as defined in the BRIEF SUMMARY OF THE INVENTION section hereinabove. In a preferred embodiment of compounds of formula III-E, R² and R³ are each CH₃.

In another preferred embodiment, in formula I-E, R¹ is 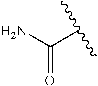
and C5 has S stereochemistry, corresponding to a compound represented by formula IV-E

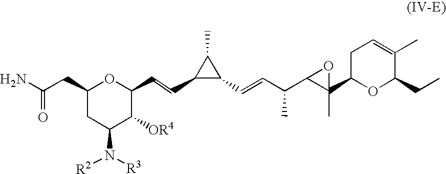

where R², R³ and R⁴ are as defined in the BRIEF SUMMARY OF THE INVENTION section hereinabove.

In other preferred embodiments, R¹ is

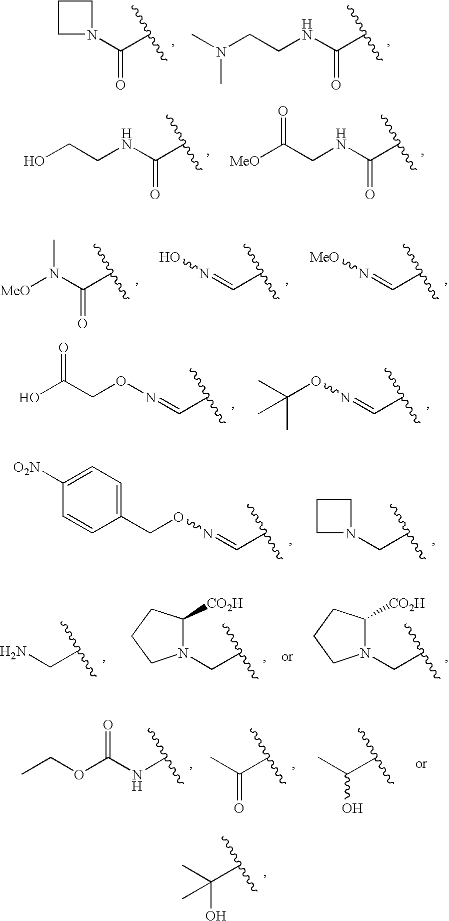

Some other preferred embodiments of R² and R³ in formulae I, II, III, VI, I-A, II-A, III-A, IV-A, I-B, II-B, III-B, IV-B, I-C, II-C, III-C, IV-C, I-D, II-D, III-D, IV-D, I-E, II-E, III-E, and/or IV-E are now disclosed. In one embodiment, R² is H, CH₃, aryl(CH₂), cycloalkyl(CH₂), or cycloalkyl; and R³ is C₂-C₅ alkyl, aryl(CH₂), cycloalkyl(CH₂), or cycloalkyl.

In another preferred embodiment, R² is H, CH₃, CH₃CH₂, HOCH₂CH₂,

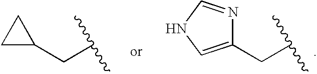

In another preferred embodiment, R³ is CH₃CH₂, CH₂CH₂OH, (CH₃)₂CH, CH₃CH₂CH₂, CH₃CH₂CH₂CH₂, COCF₃, CH₂CH₂F₂CH₂CHF₂, CH₂CF₃,

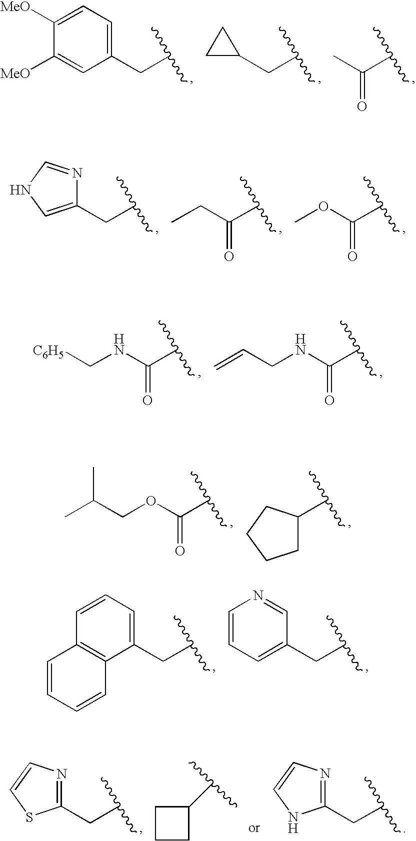

In yet another preferred embodiment, R³ is CH₃CH₂, CH₂CH₂OH, (CH₃)₂CH, CH₃CH₂CH₂, CH₃CH₂CH₂CH₂,

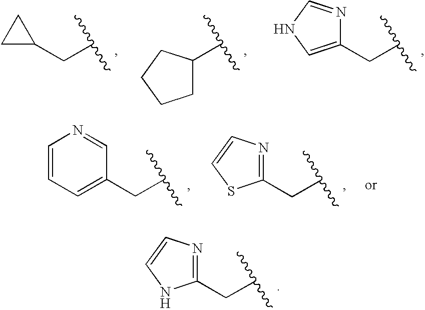

In one embodiment, R² and R³ together are CH₂CH₂CH₂

In another preferred embodiment, R² and R³ are the same but each is other than H or CH₃ when R¹ is 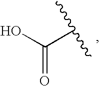
R¹⁰ and R¹¹ are both CH₃, X¹ is a bond, X² and X³ are a bond, and R² is H or CH₃, then R³ is other than H or CH₃.

In another preferred embodiment, R¹ is CO₂H, R² is CH₃ or CH₃CH₂, R⁴ is H, and R³ is selected from the group consisting of CH₃CH₂, HOCH₂CH₂, (CH₃)₂CH,

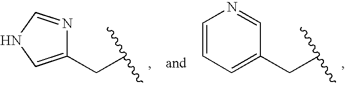

The present invention includes within its scope prodrugs of the compounds of this invention. Such prodrugs are in general functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to a subject in need thereof. Conventional procedures for the selection and preparation of suitable prodrug derivatives are described, for example, in Wermuth, “Designing Prodrugs and Bioprecursors,” in Wermuth, ed., The Practice of Medicinal Chemistry, 2nd Ed., pp. 561-586 (Academic Press 2003). Prodrugs include esters that hydrolyze in vivo (for example in the human body) to produce a compound of this invention or a salt thereof. Suitable ester groups include, without limitation, those derived from pharmaceutically acceptable aliphatic carboxylic acids, particularly alkanoic, alkenoic, cycloalkanoic and alkanedioic acids, in which each alkyl or alkenyl moiety preferably has no more than six carbon atoms. Illustrative esters include formates, acetates, propionates, butyrates, acrylates, citrates, succinates, and ethylsuccinates.

Unless particular stereoisomers are specifically indicated (e.g., by a bolded or dashed bond at a relevant stereocenter in a structural formula, by depiction of a double bond as having E or Z configuration in a structural formula, or by use stereochemistry-designating nomenclature), all stereoisomers are included within the scope of the invention, as pure compounds as well as mixtures thereof. Unless otherwise indicated, individual enantiomers, diastereomers, geometrical isomers, and combinations and mixtures thereof are all encompassed by the present invention. Polymorphic crystalline forms and solvates are also encompassed within the scope of this invention.

The present invention also includes compounds of this invention in an isolated or purified form.

Exemplary compounds of this invention are shown in Table A (the stereochemistry at C5 being S, except for compound I-kkk which is a mixture of C5 R and S epimers):

TABLE A
Compounds According to Formula I-A

| Compound | R¹ | R² | R³ | R⁴ |
|---|---|---|---|---|
| I-a | 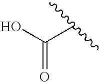 | CH₃ | CH₃CH₂ | H |
| I-b | 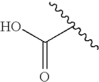 | CH₃ | 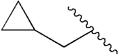 | H |
| I-c | 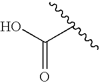 | CH₃ | 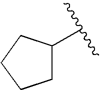 | H |
| I-d | 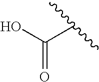 | H | 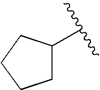 | H |
| I-e | 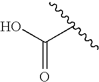 | 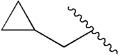 | 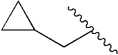 | H |
| I-f | 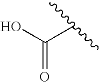 | CH₃ | 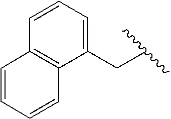 | H |
| I-g | 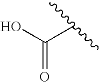 | 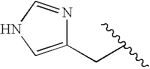 | 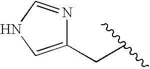 | H |
| I-h | 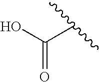 | CH₃ | 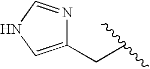 | H |
| I-i | 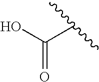 | CH₃ | 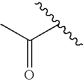 | H |
| I-j | 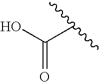 | H | 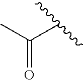 | H |
| I-k | 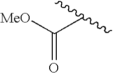 | CH₃ | 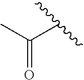 | H |
| I-l | 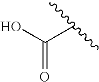 | H | 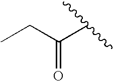 | H |
| I-m | 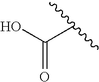 | CH₃ | 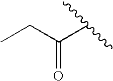 | H |
| I-n | 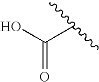 | CH₃ | HOCH₂CH₂ | H |
| I-o | 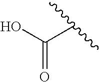 | CH₃ | 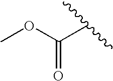 | H |
| I-p | 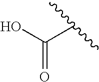 | CH₃ | 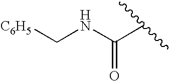 | H |
| I-q | 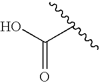 | CH₃ | 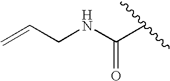 | H |
| I-r | 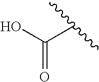 | CH₃ | 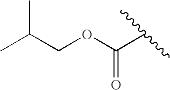 | H |
| I-s | 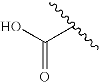 | H | 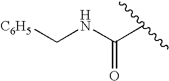 | H |
| I-t | 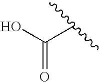 | CH₃ | CH₃CH₂CH₂ | H |
| I-u | 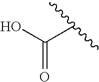 | CH₃ | CH₃CH₂CH₂CH₂ | H |
| I-v | 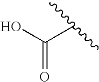 | CH₃ | (CH₃)₂CH | H |
| I-w | 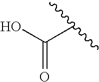 | CH₃ | 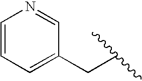 | H |
| I-x | 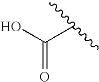 | CH₃ | 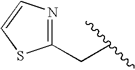 | H |
| I-y | 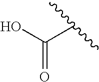 | CH₃ | 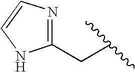 | H |
| I-z | 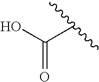 | CH₃CH₂ | CH₃CH₂ | H |
| I-aa | 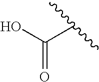 | HOCH₂CH₂ | HOCH₂CH₂ | H |
| I-bb | 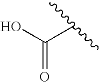 | H | 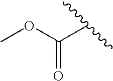 | H |
| I-cc | 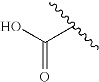 | CH₃ | 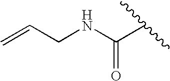 | 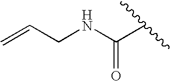 |
| I-dd | 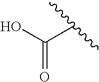 | CH₃ | 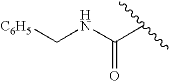 | 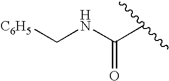 |
| I-ee | 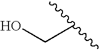 | H | CH₃ | H |
| I-ff | 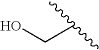 | CH₃ | CH₃ | H |
| I-gg | 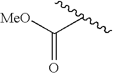 | CH₃ | 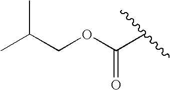 | H |
| I-hh | 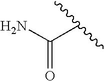 | CH₃ | CH₃ | H |
| I-ii | 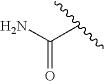 | CH₃ | CH₃ | 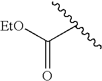 |
| I-jj | 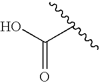 | H | 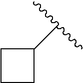 | H |
| I-kk | 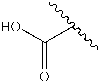 | H | (CH₃)₂CH | H |
| I-ll | 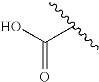 | H | HOCH₂CH₂ | H |
| I-mm | 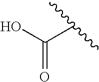 | H | CH₃CH₂ | H |
| I-nn | 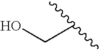 | CH₃ | HOCH₂CH₂ | H |
| I-oo | 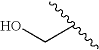 | CH₃ | (CH₃)₂CH | H |
| I-pp | 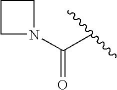 | CH₃ | CH₃ | H |
| I-qq | 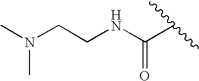 | CH₃ | CH₃ | H |
| I-rr | 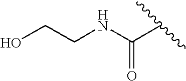 | CH₃ | CH₃ | H |
| I-ss | 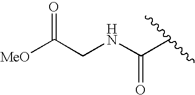 | CH₃ | CH₃ | H |
| I-tt | 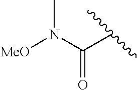 | CH₃ | CH₃ | H |
| I-uu | 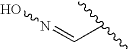 | CH₃ | CH₃ | H |
| I-vv | 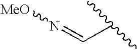 | CH₃ | CH₃ | H |
| I-ww | 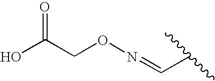 | CH₃ | CH₃ | H |
| I-xx | 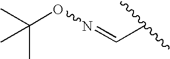 | CH₃ | CH₃ | H |
| I-yy | 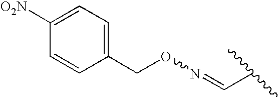 | CH₃ | CH₃ | H |
| I-zz | 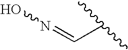 | CH₃ | H | H |
| I-aaa | 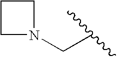 | CH₃ | CH₃ | H |
| I-bbb | 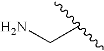 | CH₃ | CH₃ | H |
| I-ccc | 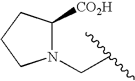 | CH₃ | CH₃ | H |
| I-ddd | 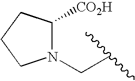 | CH₃ | CH₃ | H |
| I-eee | 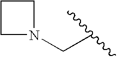 | H | H | H |
| I-fff | 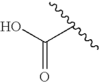 | H | COCF₃ | H |
| I-ggg | 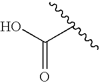 | H | CH₂CHF₂ | H |
| I-hhh | 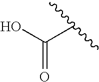 | H | 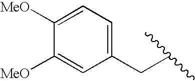 | H |
| I-iii | 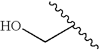 | H | CH₂CF₃ | H |
| I-jjj | 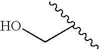 | H | CH₂CHF₂ | H |
| I-kkk | 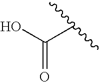 | R² and R³ together are CH₂CH₂CH₂ | | H |
| I-lll | 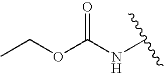 | CH₃ | CH₃ | H |
| I-mmm | 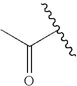 | CH₃ | CH₃ | H |
| I-nnn | 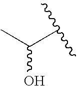 | CH₃ | CH₃ | H |
| I-ooo | 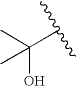 | CH₃ | CH₃ | H |

TABLE B
Compounds According to Formula I-B

| Compound | R¹ | R² | R³ | R⁴ |
|---|---|---|---|---|
| II-a | 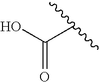 | CH₃ | CH₃ | H |
| II-b | 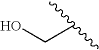 | CH₃ | CH₃ | H |

TABLE C
Compound According to Formula I-C

| Compound | R¹ | R² | R³ | R⁴ |
|---|---|---|---|---|
| III-a | 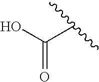 | CH₃ | CH₃ | H |

TABLE D
Compounds According to Formula I-D

| Compound | R¹ | R² | R³ | R⁴ |
|---|---|---|---|---|
| IV-a | 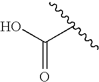 | H | H | H |
| IV-b | 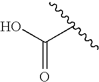 | CH₃ | CH₃ | H |
| IV-c | 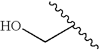 | CH₃ | CH₃ | H |
| IV-d | 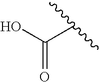 | CH(CH₃)₂ | H | H |
| IV-e | 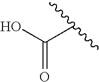 | H | CH₃ | H |

TABLE E
Compounds According to Formula I-E

| Com- pound | R¹ | R² | R³ | R⁴ | 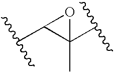 |
|---|---|---|---|---|---|
| V-a | 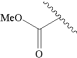 | 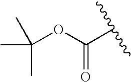 | H | H | 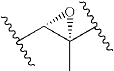 |
| V-b | 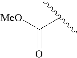 | 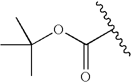 | H | H | 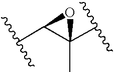 |
| V-c | 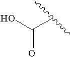 | H | H | H | 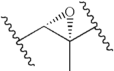 |
| V-d | 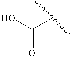 | H | H | H | 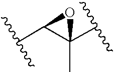 |

It is understood that compounds of formula I having both a carboxylic acid and an amine group may exist in a zwitterionic form. Formula I is intended to embrace such zwitterionic forms.

Compounds of this invention are also useful as synthons for preparing other ambruticin derivatives or analogs, having superior antifungal properties.

This invention also provides for the method such that the growth of a fungal cell is inhibited. The inhibition of the cell comprises a reduction in the growth of the cell. The reduction of growth includes one or more of the following: a decrease in the growth of the cell, a decrease in the rate of cell division of the cell, and the killing of the cell. This invention also provides for the method such that the subject is cleared of a fungal infection, or is relieved of a symptom caused by the fungal infection. This invention also provides for the method such that the subject, who but for the administering of the pharmaceutical composition to the subject avoids a fungal infection. In one embodiment, the fungal infection is a pulmonary, skin, central nervous system, systemic or invasive infection.

In one embodiment, the subject in need of such treatment or reduction of probability of the fungal infection is one who has a high chance of acquiring the fungal infection. Such subjects include healthy or immunocompromised individuals.

The fungal cell or the fungal cell causing the fungal infection is any fungal cell that is susceptible to ambruticin or an ambruticin analog. The fungal cell or the fungal cell causing the fungal infection can be any species of the following genera: Aspergillus, Blastomyces, Candida, Coccidiodes, Crytococcus, Epidermophyton, Fusarium, Hansenula, Histoplamsma, Microsporium, Mucor, Pseudallescheria, Rhizopus, Scedosporium, and Trichophyton.

In one embodiment, the Aspergillus species is Aspergillus fumigatus, Aspergillus flavus, Aspergillus niger, Aspergillus nidulans, Aspergillus terreus, Aspergillus clavatus, Aspergillus glaucus, or Aspergillus versicolor. Aspergillus fumigatus strains include strain ATCC 204305. Aspergillus flavus strains include strain ATCC 204304. In one embodiment, the cell of the genus Aspergillus is a strain that is resistant to one or more antibiotics, wherein none is an ambruticin. Such antibiotics include, but are not limited, voriconazole, amphotericin B (deoxycholate and lipid preparations), itraconazole posaconazole, ravuconazole, caspofungin, FK463, and anidulafungin (LY303366).

In one embodiment, the Blastomyces species is Blastomyces dermatitidis.

In one embodiment, the Candida species is Candida parapsilosis or Candida dublinensis. Of the ambruticin analogs tested, none were found to sufficiently inhibit certain Candida albicans species. However, certain strain(s) of Candida albicans are susceptible to ambruticin compounds; for example, compound IV-a inhibits growth of Candida albicans strain 05-1422 (see Table T). In one embodiment, the fungal ingection is caused by a Candida species or Candida strain (such as C. albicans strain such as strain 05-1422) that is susceptible to a compound of this invention. In one embodiment, the fungal infection is candidemia or candidiasis.

In one embodiment, the Coccidiodes species is Coccidioides immitis or Coccidioides posadasii. Coccidioides immitis strains include strains Silveira, 46, ATCC 7366, K9-71X, 98-449, 98-571, Kr, DA, Ma, Mc, Co, Si, In, La, Sy, and Ro (González et al. Antimicrob. Agents Chemother. 45(6):1854-1859 (2001); Rifkind et al., Antimicrob. Agents Chemother. 6(6):783-784 (1974); Ward et al., Infect Immun. 12(5):1093-1097 (1975)).

In one embodiment, the Crytococcus species is Cryptococcus neoformans, Cryptococcus albidus var. albidus, Cryptococcus albidus var. diffluens, Cryptococcus luteolus, Cryptococcus laurentii, Cryptococcus uniguttulatus, Cryptococcus terreus, or Cryptococcus gastricus. In a preferred embodiment, Cryptococcus neoformans is Cryptococcus neoformans var. neoformans, Cryptococcus neoformans var. gattii, or Cryptococcus neoformans var. grubii. In one embodiment, the Cryptococcus neoformans is strain 97-14, 11239, or 11240. In one embodiment, the cell of the genus Cryptococcus is a strain that is resistant to one or more antibiotics, wherein none is an ambruticin. Such antibiotics include, but are not limited to, fluconazole, amphotericin B (deoxycholate and lipid preparations), itraconazole, and 5-flurocytosine.

In one embodiment, the Epidermophyton species is Epidennophyton floccosum.

In one embodiment, the Fusarium species is Fusarium solani.

In one embodiment, the Hansenula species is Hansenula anomala.

In one embodiment, the Histoplasma species is Histoplasma capsulatum. In one embodiment, the fungal infection is histoplasmosis.

In one embodiment, the Microsporium species is Microsporum gypseum or Microsporum canis.

In one embodiment, the Pseudallescheria species is Pseudallescheria boydii.

In one embodiment, the Rhizopus species is Rhizopus oryzei.

In one embodiment, the Scedosporium species is Scedosporium apiospermum or Scedosporium prolificans.

In one embodiment, the Trichophyton species is Trichophyton mentagrophytes, Trichophyton interdigitale, or Trichophyton rubrum.

In one embodiment, the fungal infection is an Aspergillus infection or aspergillosis. In one embodiment, the aspergillosis is allergic bronchopulmonary aspergillosis, pulmonary aspergilloma, or invasive aspergillosis. In one embodiment, the site of the allergic bronchopulmonary aspergillosis is in one or more of the following: sinuses and lungs. In one embodiment, the site of the pulmonary aspergilloma is in a lung cavity. In one embodiment, the invasive aspergillosis is one or more of the following infections: pulmonary aspergillosis, central nervous system (CNS) aspergillosis, sinonasal aspergillosis, osteomyelitis, endophthalmitis, endocarditis, renal abscess, and cutaneous infection. In one embodiment, the Aspergillus infection is cutaneous (resulting from a trauma, such as a burn, a post-surgical wound, or a intravenous insertion site), otomycosis, exogenous endophthalmitis, allergic fungal sinusitis, or a urinary infection. In one embodiment, the site of Aspergillus infection is in the subject's respiratory system. In one embodiment, the site of Aspergillus infection is in the subject's lungs. In another embodiment, the site of Aspergillus infection is in the subject's gastrointestinal tract, brain, liver, kidney, heart, skin, and/or eye. In one embodiment, the aspergillosis that is an allergic form of aspergillosis, non-invasive colonization aspergillosis, or invasive aspergillosis. In one embodiment, the allergic form of aspergillosis is asthma, allergic bronchopulmonary aspergillosis, or extrinsic allergic alveolitis. In one embodiment, non-invasive colonization aspergillosis is aspergilloma or a non-pulmonary local infection. In one embodiment, invasive aspergillosis is pulmonary or disseminated.

In one embodiment, the fungal infection is Cryptococcus infection or cryptococcosis. In one embodiment, the cryptococcosis is localized or disseminated. In one embodiment, the localized cryptococcosis is a pulmonary cryptococcosis. The pulmonary cryptococcosis is an acute infection or is chronic. The disseminated cryptococcosis is acute or chronic. In one embodiment, the cryptococcosis is cryptococcal meningitis. The site of the Cryptococcus infection can be in the CNS, or in the respiratory system, such as in the lungs.

In one embodiment, the fungal infection is Coccidioides infection or coccidioidomycosis (also known as Valley Fever or Desert Fever). In one embodiment, the site of Coccidioides infection is in the subject's respiratory system. In one embodiment, the site of Coccidioides infection is in the subject's lungs. In another embodiment, the site of Coccidioides infection is in the subject's kidneys, spleen, lymph nodes, brain, blood, and/or thyroid gland. In one embodiment, the subject is suffering from coccidioidomycosis that is asymptomatic, acute symptomatic, or chronic pulmonary. Acute symptomatic coccidioidomycosis can have one or more of the following symptoms: pulmonary syndrome combined with cough, chest pain, shortness of breath, fever, and/or fatigue; diffuse pneumonia; skin manifestations (such as fine papular rash, erythema nodosum, and erythema multiforme); migratory arthralgias; and, fever. Chronic pulmonary coccidioidomycosis can have one or more of the following symptoms: pulmonary nodules and peripheral thin-walled cavities. In another embodiment, the subject is suffering from coccidioidomycosis that is extrapulmonary or disseminated. Coccidioidomycosis that is extrapulmonary or disseminated has one or more of the following symptoms: keratotic ulcers; verrucose ulcers; subcutaneous fluctuant abscesses; synovitis and effusion affecting the knees, wrists, feet, ankles, and/or pelvis; lytic lesions affecting the axial skeleton; meningeal disease; and, infection of the thyroid, gastrointestinal tract, adrenal glands, genitourinary tract, pericardium, and/or peritoneum. In one embodiment, the subject is suffering from coccidioidal meningitis.

In one embodiment, the subject is one who is diagnosed with a fungal infection, and/or is immunocompromised. Examples of immunocompromised subjects include, but are not limited to, patients infected with Human Immunodeficiency Virus, organ transplant recipients, patients undergoing chemotherapy (such as cancer patients), patients undergoing corticosteroids therapy, cancer patients, patients with diabetes mellitus, chronic obstructive pulmonary disease, cirrhosis, rheumatoid arthritis, or systemic lupus erythematous, pregnant women, and patients having undergone a splenectomy. Cancer patients include, but are not limited to, patients afflicted with chronic lymphatic leukemia, Hodgkin's disease, chronic myelogenous leukemia, myeloma, lymphosarcoma, acute lymphoblastic leukemia, or lung cancer.

In another embodiment, the subject in need of such treatment or reduction of probability of fungal infection is one who has a high chance of acquiring fungal infection. Such subjects include healthy or immunocompromised individuals. For example, such subjects may be or are going to travel to or through an area where coccidioidomycosis is endemic.

Modes of Administration and Pharmaceutical Formulations

Suitable modes of administration of the pharmaceutical composition include, but are not limited to, oral, topical, aerosol, inhalation by spray, parenteral, subcutaneous, intravenous, intramuscular, interperitoneal, rectal, and vaginal administration. The term parenteral, as used herein, includes subcutaneous injections, and intravenous, intrathecal, intramuscular, and intrasternal injection or infusion techniques. A preferred mode of administration is one that brings a compound of this invention to the actual or potential site(s) of fungal infection in the subject. The pharmaceutical composition can be in a solid, semi-solid, or liquid form

The pharmaceutically acceptable carriers described herein, for example, vehicles, adjuvants, excipients, and diluents, are well known to those who are skilled in the art and are readily available. Preferably, the carrier is chemically inert to a compound of this invention and has no detrimental side effects or toxicity under the conditions of use. Preferably, the pharmaceutically acceptable carrier is free of pyrogen. The pharmaceutically acceptable carriers which can be used include, but are not limited to, water, glucose, lactose, gum acacia, gelatin, mannitol, starch paste, magnesium trisilicate, talc, corn starch, keratin, colloidal silica, potato starch, and urea.

The amount of a compound of this invention that may be combined with the pharmaceutically acceptable carrier to produce a single dosage form will vary depending upon the subject treated and the particular mode of administration. Suitable dosage levels of the active ingredient are of the order from about 0.01 mg to about 100 mg per kg body weight per day, preferably from about 0.1 mg to about 50 mg per kg body weight per day. Dosage unit forms will generally contain from about 0.1 mg to about 500 mg of the active ingredient. For external administration, the active ingredient may be formulated within the range of, for example, 0.00001% to 60% by weight, and preferably from 0.001% to 10% by weight. In addition, the pharmaceutical composition can be administered on an intermittent basis, i.e., at daily, semi-weekly, or weekly intervals. It will be understood, however, that the specific dose level for a particular subject will depend on a variety of factors. These factors include the activity of the specific compound employed; the age, body weight, general health, sex, and diet of the subject; the time and route of administration and the rate of excretion of the drug; whether a drug combination is employed in the treatment; and, the severity of the particular disease or condition for which therapy is sought.

The pharmaceutical compositions suitable for oral administration include, but are not limited to, (a) liquid formulations; (b) capsules, sachets, tablets, lozenges, and troches, each containing a predetermined amount of the active ingredient, as solids or granules; (c) powders; (d) suspensions; and (e) suitable emulsions. Liquid formulations may include diluents, such as water and alcohols, and optionally a pharmaceutically acceptable surfactant. Capsule forms can be of the ordinary hard- or soft-shelled gelatin type containing, for example, surfactants, lubricants, and inert fillers. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and the like. The tablet can further comprise one or more colorants, diluents, buffering agents, disintegrating agents, moistening agents, preservatives, or flavoring agents.

The pharmaceutical composition, alone or in combination with other suitable components, can be made into aerosol formulations to be administered via inhalation. These aerosol formulations can be placed into pressurized acceptable propellants (such as dichlorodifluoromethane, propane, nitrogen, and the like) or non-pressured preparations (such as in a nebulizer or an atomizer). When the site(s) of infection of a subject is the lungs, a preferred mode of administration is inhalation of an aerosol formulation either orally or nasally. Preferably, the aerosol formulation comprises particles of a respirable size, including, but not limited to, mean particle sizes of 5 μm to 500 μm.

The pharmaceutical composition can be an injectable formulation. The requirements for effective carriers for injectable compositions are well known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., pages 622-630 (1986)). Preferably, injectable compositions are administered intravenously. Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The pharmaceutical composition can further comprise an excipient. Excipients that may be used include one or more carriers, surface active agents, thickening or emulsifying agents, solid binders, dispersion or suspension aids, solubilizers, colorants, flavoring agents, coatings, disintegrating agents, lubricants, sweeteners, preservatives, isotonic agents, and combinations thereof. The selection and use of suitable excipients is taught in Gennaro, ed., Remington: The Science and Practice of Pharmacy, 20th Ed. (Lippincott Williams & Wilkins 2003), the disclosure of which is incorporated herein by reference.

The present invention provides for an isolated or recombinant cell comprising the genes of the ambruticin biosynthetic gene cluster and producing one or more ambruticins or ambruticin analogs, wherein the activity the ambP, ambO, ambS, or ambM gene product is reduced or disrupted. In one embodiment, the reduction of activity is due to the reduced expression of the gene encoding the gene product, or the gene product is modified so that there is less or no activity. In one embodiment, the ambruticin gene is disrupted due the mutation of the gene such that that the gene product of the ambruticin gene is not expressed. In one embodiment, one or more of the ambruticins or ambruticin analogs, which is produced by the complete wild type ambruticin gene cluster, is not produced. In a preferred embodiment, the gene is entirely or partially deleted. The gene of interest can be disrupted or deleted by transposon insertion, homologous recombination, mutagenesis using a mutagen, or the like.

In one embodiment, the isolated or recombinant cell, wherein the activity of the ambM gene product is reduced or disrupted, produces an ambruticin analog in which R¹⁰ is H. In one embodiment, the isolated or recombinant cell, wherein the activity of the ambP and/or ambO gene product(s) is/are reduced or disrupted, produces an ambruticin analog in which X² and X³ are each H. In one embodiment, the isolated or recombinant cell, wherein the activity of the ambS gene product is reduced or disrupted, produces elevated amounts of ambruticin VS-5 and ambruticin S and does not produce ambruticin VS-1, ambruticin VS-2, ambruticin VS-3 and ambruticin VS-4.

In one embodiment, the isolated or recombinant cell is disrupted for the ambS gene and, when cultured, the cell produces ambruticin VS-5 and ambruticin S, and does not produce ambruticin VS-3 or ambruticin VS-4. Preferably, the cell overproduces ambruticin VS-5 and ambruticin S as compared to a cell that is not disrupted for the ambS gene.

In one embodiment, the isolated or recombinant cell is disrupted for the ambP or ambO gene, or both genes; and, when cultured, the cell produces 20,21-dihydro analogs of the ambruticin, for example, compounds IV-a and IV-b, and compound II-D wherein R² is CH₃ and R³ is H.

In one embodiment, the isolated or recombinant cell is disrupted for the ambM gene and when cultured, the cell produces ambruticin lacking the C27 methyl group, for example, compound III-a and compound II-B wherein R² is H or CH₃ and R³ is H.

The present invention provides for an isolated or recombinant cell comprising the genes of the ambruticin biosynthetic gene cluster, wherein the malonate specific AT domain from module 7 is replaced or engineered into a loading domain. The loading domain can be any suitable loading domain of any suitable polyketide synthase (PKS). Preferably, the loading domain is one derived or obtained from an ambruticin PKS gene or ambruticin gene cluster. The isolated or recombinant cell produces an ambruticin analog in which R¹¹ is H. In one embodiment, the isolated or recombinant cell, wherein the malonate specific AT domain from module 7 is replaced or engineered into a loading domain, produces an ambruticin analog in which R¹¹ is H.

In one embodiment, the isolated or recombinant cell lacks the activities of two or more of the ambP, ambO, ambS, or ambM gene products.

In another embodiment, the cell is deleted for the ambM gene and the malonate specific AT domain from module 7 is replaced or engineered into a loading domain. The cell produces ambruticin S which lacks the C24 and C27 methyl groups, and the compounds as represented by compound (II), wherein R¹⁰ and R¹¹ are each H, X is a bond, X² and X³ are together a bond, and R² and R³ are independtly H or CH₃.

The present invention provides for a method of producing one or more ambruticins or ambruticin analogs comprising culturing the isolated or recombinant cell comprising the genes of the ambruticin gene cluster and producing one or more ambruticins or ambruticin analogs, wherein the activity the ambP, ambO, ambS, or ambM gene product is reduced or disrupted, or the malonate specific AT domain from module 7 is replaced or engineered into a loading domain, or a combination thereof. In one embodiment, the method further comprises purifying the ambruticins or ambruticin analogs.

In one aspect, the cell is native to the ambruticin biosynthetic gene cluster. Alternatively, the cell is a host cell that is either native or heterologous to the ambruticin gene cluster, wherein the ambruticin biosynthetic genes are present, either on a vector or integrated into the chromosome of the cell. A cell native to the ambruticin biosynthetic gene cluster is a cell of the genus Sorangium. Preferably, the cell is a Sorangium cellulosum. More preferably, the cell is the So ce10, NCIMB12601 or So ce307 strain of Sorangium cellulosum. A host cell heterologous to the ambruticin gene cluster includes, but is not limited to, eubacterial cells such as E. coli, yeast cells such as Saccharomyces cerevisiae, or myxobacterial cells such as Myxococcus xanthus. U.S. patent application Ser. No. 11/075,185 and WO 2005/086907, each incorporated herein by reference, disclose a method for expressing ambruticin using a Myxococcus xanthus host cell.

The present invention also provides for an isolated or purified compound represented by the formula (II-D): 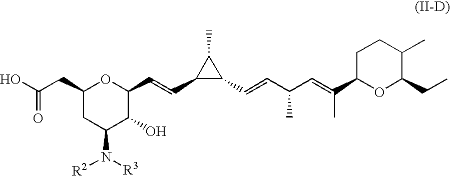
wherein R² and R³ are, independently for each occurrence thereof, H or CH₃. These compounds are also represented by compounds IV-a (20,21-dihydro ambruticin VS-5), IV-b (20,21-dihydro ambruticin VS-3) and IV-e (20,21-dihydro ambruticin VS4). Compounds IV-a, IV-b, and IV-e are produced by the cell described above that lack the activity of the ambO and/or ambP gene product(s). Preferably, the cell is deleted for the ambO and/or ambP genes. More preferably, the cell is Sorangium cellulosum So ce10. In one embodiment the cell is cultured and the compounds of interest are isolated or purified using methods previously described (see Examples 22 and 23; and U.S. patent application Ser. No. 11/075,185 and WO 2005/086907, each incorporated herein by reference)). These methods can be used to produce and purify other ambruticin compounds and analogs disclosed in this specification.

In another embodiment, the cell is deleted for the ambO and/or ambP genes, and is also deleted for the ambS gene. The cell produces compound IV-a and 20,21-dihydro ambruticin S.

In another embodiment, the cell is deleted for the ambO and/or ambP genes, and is also deleted for the ambM gene. The cell produces 20,21-dihydro ambruticin S which lacks the C27 methyl group, and the compounds as represented by compound (II), wherein R¹⁰ is H, R¹¹ is CH₃, X¹ is a bond, X² and X³ are each H, and R² and R³ are independtly H or CH₃.

In another embodiment, the cell is deleted for the ambO and/or ambP genes, and the malonate specific AT domain from module 7 is replaced or engineered into a loading domain. The cell produces 20,21-dihydro ambruticin S which lacks the C24 methyl, and the compounds as represented by compound (II), wherein R¹⁰ is CH₃, R¹¹ is H, X¹ is a bond, X² and X³ are each H, and R² and R³ are independtly H or CH₃.

In another embodiment, the cell is deleted for the ambM and ambO and/or ambP genes, and the malonate specific AT domain from module 7 is replaced or engineered into a loading domain. The cell produces 20,21-dihydro ambruticin S which lacks the C24 methyl, and the compounds as represented by compound (II), wherein R¹⁰ and R¹ are each H, X¹ is a bond, X² and X³ are each H, and R² and R³ are independtly H or CH₃.

The practice of this invention can be further understood by reference to the following examples, which are provided by way of illustration and not of limitation.

### EXAMPLE 1

Compounds I wherein R¹ is CO₂H; R² is CH₃; R³ is alkyl, cycloalkyl, etc.; and R⁴ is H were prepared from ambruticin VS-4 per the following equation:

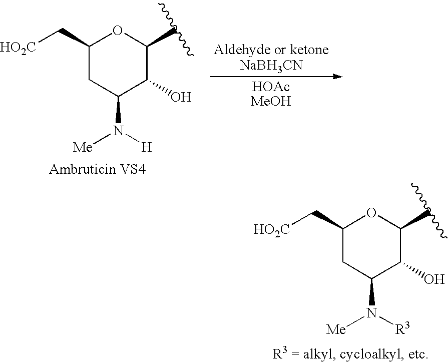

The following general procedure was used: To a solution of ambruticin VS-4 ((5S,6R)-5-(methylamino)-6-hydroxypolyangioic acid, 0.1 mmol) in methanol (1 mL) was added the aldehyde or ketone (0.2 mmol) and acetic acid (0.4 mmol), followed by sodium cyanoborohydride (0.2 mmol). The solution was stirred at 20 to 25° C. (for reactive aldehydes) or 50 to 60° C. (for less reactive aldehydes and ketones) until all of the ambruticin VS-4 was consumed. The reaction mixture was concentrated on a rotary evaporator, re-dissolved in a mixture of water-acetonitrile, filtered through a one-gram plug of C-18 silica gel, and purified by reversed-phase HPLC, eluted using a gradient of acetonitrile in water containing 0.1% acetic acid. The product was obtained as a white solid after lyophilization of desired fractions.

Compound I-a ((5S,6R)-5-(N-ethyl-methylamino)-6-hydroxypolyangioic acid) was synthesized using acetaldehyde at room temperature. ESI-TOF-MS m/z 516.3701, calcd for C₃₁H₅₀NO₅ ([M+H]⁺) 516.3684.

Compound I-b ((5S,6R)-5-(N-cyclopropylmethyl-methylamino)-6-hydroxypolyangioic acid) was synthesized using cyclopropanecarboxaldehyde at room temperature. ESI-TOF-MS m/z 542.3828, calcd for C₃₃H₅₂NO₅ ([M+H]⁺) 542.3840.

Compound I-c ((5S,6R)-5-(N-cyclopentyl-methylamino)-6-hydroxypolyangioic acid) was synthesized using cyclopentanone at 50° C. ESI-TOF-MS m/z 556.3981, calcd for C₃₄H₅₄NO₅ ([M+H]⁺) 556.3996.

Compound I-f ((5S,6R)-5-(N-(2-naphthyl)methyl-methylamino)-6-hydroxypolyangioic acid) was synthesized using 2-naphthaldehyde at room temperature. ESI-TOF-MS m/z 628.3967, calcd for C₄₀H₅₄NO₅ ([M+H]⁺) 628.3996.

Compound I-h ((5S,6R)-5-(N-(4-imidazolyl)methyl-methylamino)-6-hydroxypolyangioic acid) was synthesized using 4-imidazolecarboxaldehyde at room temperature.

Compound I-n ((5S,6R)-5-(N-(2-hydroxyethyl)-methylamino)-6-hydroxypolyangioic acid) was synthesized using glycolaldehyde dimer at room temperature. ESI-TOF-MS m/z 532.3638, calcd for C₃₁H₅₀NO₆ ([M+H]⁺) 532.3633.

Compound I-t ((5S,6R)-5-(N-propyl-methylamino)-6-hydroxypolyangioic acid) was synthesized using propionaldehyde at room temperature. ESI-TOF-MS m/z 530.3838, calcd for C₃₂H₅₂NO₅ ([M+H]⁺) 530.3840.

Compound I-u ((5S,6R)-5-(N-butyl-methylamino)-6-hydroxypolyangioic acid) was synthesized using butyraldehyde at room temperature. ESI-TOF-MS m/z 544.3969, calcd for C₃₃H₅₄NO₅ ([M+H]⁺) 544.3997.

Compound I-v ((5S,6R)-5-(N-isoproyl-methylamino)-6-hydroxypolyangioic acid) was synthesized using acetone at 50° C. ESI-TOF-MS m/z 530.3841, calcd for C₃₂H₅₂NO₅ ([M+H]⁺) 530.3840.

Compound I-w ((5S,6R)-5-(N-(3-pyridyl)methyl-methylamino)-6-hydroxypolyangioic acid) was synthesized using nicotinaldehyde at 50° C. ESI-TOF-MS m/z 579.3827, calcd for C₃₅H₅₁N₂O₅ ([M+H]⁺) 579.3893.

Compound I-x ((5S,6R)-5-(N-(2-thiazolyl)methyl-methylamino)-6-hydroxypolyangioic acid) was synthesized using 2-thiazolecarboxaldehyde at 60° C. ESI-TOF-MS m/z 585.3341, calcd for C₃₃H₄₉N₂O₅ ([M+H]⁺) 585.3357.

Compound I-y ((5S,6R)-5-(N-(2-imidazolyl)methyl-methylamino)-6-hydroxypolyangioic acid) was synthesized using 2-imidazolecarboxaldehyde at 60° C. ESI-TOF-MS m/z 568.3719, calcd for C₃₃H₅₀N₃O₅ ([M+H]⁺) 568.3745.

### EXAMPLE 2

Compounds I wherein R¹ is CO₂H; R² and R³ are H, alkyl, etc.; and R⁴ is H were prepared from ambruticin VS-5 per the following equation:

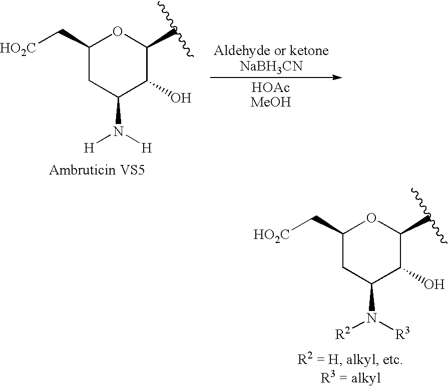

The following general procedure was used: To a solution of ambruticin VS-5 ((5S,6R)-5-amino-6-hydroxypolyangioic acid, 0.1 mmol) in methanol (1 mL) was added the aldehyde or ketone (0.2 mmol) and acetic acid (0.4 mmol), followed by sodium cyanoborohydride (0.2 mmol). The solution was stirred at 20 to 25° C. until all of the ambruticin VS-5 was consumed. The reaction mixture was concentrated on a rotary evaporator, re-dissolved in a mixture of water-acetonitrile, filtered through a one-gram plug of C-18 silica gel, and purified by reversed-phase HPLC, eluted using a gradient of acetonitrile in water containing 0.1% acetic acid. The product was obtained as a white solid after lyophilization of desired fractions.

Compound I-d ((5S,6R)-5-(cyclopentylamino)-6-hydroxypolyangioic acid) was synthesized using cyclopentanone at room temperature. ESI-TOF-MS m/z 542.3848, calcd for C₃₃H₅₂NO₅ ([M+H]⁺) 542.3840.

Compound I-e ((5S,6R)-5-[di(cyclopropylmethyl)amino]-6-hydroxypolyangioic acid) was synthesized using cyclopropanecarboxaldehyde. ESI-TOF-MS nz/z 582.4163, calcd for C₃₆H₅₆NO₅ ([M+H]⁺) 582.4153.

Compound I-g ((5S,6R)-5-{di [(4-imidazolyl)methyl]amino}-6-hydroxypolyangioic acid) was synthesized using 4-imidazolecarboxaldehyde. ESI-TOF-MS m/z 634.3966, calcd for C₃₆H₅₂N₅O₅ ([M+H]⁺) 634.3963.

Compound I-z ((5S,6R)-5-(diethylamino)-6-hydroxypolyangioic acid) was synthesized using acetaldehyde. ESI-TOF-MS m/z 530.3842, calcd for C₃₂H₅₂NO₅ ([M+H]⁺) 530.3840.

Compound I-aa ((5S,6R)-5-[di(2-hydroxyethyl)amino]-6-hydroxypolyangioic acid) was synthesized using glycoaldehyde dimer.

Compound I-ggg ((5S,6R)-5-(2,2-difluoroethyl)amino-6-hydroxypolyangioic acid) was synthesized using difluoroacetaldehyde ethyl hemiacetal. ESI-TOF-MS m/z 538.3337, calcd for C₃₀H₄₆F₂NO₅ ([M+H]⁺) 538.3339.

Compound I-hhh ((5S,6R)-5-(3,4-dimethoxybenzyl)amino-6-hydroxypolyangioic acid) was synthesized using 3,4-dimethoxybenzaldehyde. ESI-TOF-MS m/z 624.3887, calcd for C₃₇H₅₄NO₇ ([M+H]⁺) 542.3452.

### EXAMPLE 3

Compounds I wherein R¹ is CO₂H, R² is CH₃, R³ is acyl and R⁴ is H were prepared from ambruticin VS-4 per the following equation:

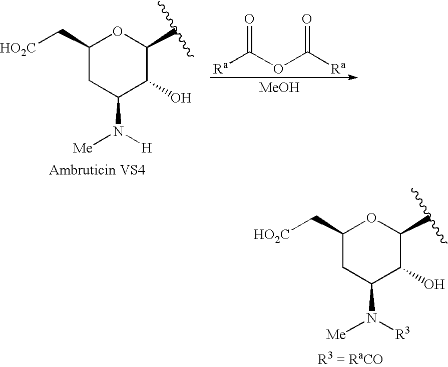

The following general procedure was used: To a solution of ambruticin VS-4 (0.1 mmol) in methanol (1 mL) was added the anhydride (1 mmol). After stirred at 20 to 25° C. for 20 h, the reaction mixture was concentrated on a rotary evaporator. The residue was re-dissolved ethyl acetate. The solution was washed with water and brine and dried over sodium sulfate. The sodium sulfate was removed by filtration and the filtrate was evaporated to dryness. The crude product was purified by reversed-phase HPLC, eluted using a gradient of acetonitrile in water containing 0.1% acetic acid. The product was obtained as a white solid after lyophilization of desired fractions.

Compound I-i ((5S,6R)-5-[N-methyl(acetamido)]-6-hydroxypolyangioic acid) was synthesized using acetic anhydride. ESI-TOF-MS m/z 530.3500, calcd for C₃₁H₄₈NO₆ ([M+H]⁺) 530.3476.

Compound I-k (methyl (5S,6R)-5-[N-methyl(acetamido)]-6-hydroxypolyangioate) was a side product in the preparation of compound I-i. ESI-TOF-MS m/z 544.3658, calcd for C₃₂H₅₀NO₆ ([M+H]⁺) 544.3633.

Compound I-m ((5S,6R)-5-[N-methyl(propionamido)]-6-hydroxypolyangioic acid) was synthesized using propionic anhydride. ESI-TOF-MS nmiz 544.3644, calcd for C₃₂H₅₀NO₆ ([M+H]⁺) 544.3633.

### EXAMPLE 4

Compounds I wherein R¹ is CO₂H, R² is H, R³ is acyl and R⁴ is H were prepared from ambruticin VS-5 per the following equation:

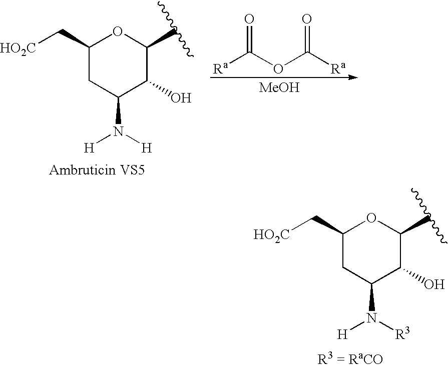

The general procedure of Example 3 was followed, except that ambruticin VS-5 was used instead of ambruticin VS-4.

Compound I-j ((5S,6R)-5-acetamido-6-hydroxypolyangioic acid) was synthesized using acetic anhydride. ESI-TOF-MS m/z 516.3339, calcd for C₃₀H₄₆NO₆ ([M+H]⁺) 516.3320.

Compound I-l ((5S,6R)-5-propionamido-6-hydroxypolyangioic acid) was synthesized using propionic anhydride. ESI-TOF-MS m/z 530.3457, calcd for C₃₁H₄₈NO₆ ([M+H]⁺) 530.3476.

Compound I-fff ((5S,6R)-5-trifluroacetamido-6-hydroxypolyangioic acid) was synthesized using trifluoroacetic anhydride in dichloromethane. ESI-TOF-MS m/z 592.2862, calcd for C₃₀H₄₂F₃NO₆Na ([M+Na]⁺) 592.2856.

### EXAMPLE 5

Compounds I wherein R¹ is CO₂H, R² is H or CH₃, R³ is R^{b}OCO and R⁴ is H were prepared from ambruticin VS-4 or VS-5 per the following equation:

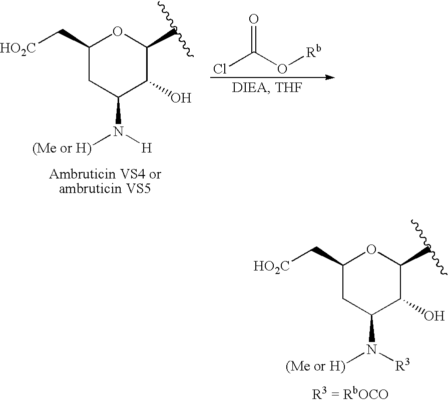

The following general procedure was used: To a suspension of ambruticin VS-4 or ambruticin VS-5 (0.1 mmol) in dry tetrahydrofuran (THF, 1 mL) was added N,N-diisopropylethylamine (DIEA, 0.3 mmol), followed by the alkyl chloroformate (0.2 mmol). After stirred at 20 to 25° C. for 20 h, the reaction mixture was concentrated on a rotary evaporator. The residue was re-dissolved in ethyl acetate. The solution was washed with 0.1 M HCl (aq) and brine, and dried over sodium sulfate. The salt was removed by filtration and the filtrate was evaporated to dryness. The crude product was purified by reversed-phase HPLC, eluted using a gradient of acetonitrile in water containing 0.1% acetic acid. The product was obtained as a white solid after lyophilization of desired fractions.

Compound I-o ((5S,6R)-5-[N-methyl(methoxycarbonylamino) was synthesized from ambruticin VS-4 and methyl chloroformate. ESI-TOF-MS m/z 568.3227, calcd for C₃₁H₄₇NO₇Na ([M+Na]⁺) 568.3248.

Compound I-bb ((5S,6R)-5-methoxycarbonylamino-6-hydroxypolyangioic acid) was synthesized from ambruticin VS-5 and methyl chloroformate. ESI-TOF-MS m/z 554.3094, calcd for C₃₀H₄₅NO₇Na ([M+Na]⁺) 554.3088.

Compound I-r ((5S,6R)-5-[N-methyl(isobutoxycarbonylamino)]-6-hydroxypolyangioic acid) was synthesized from ambruticin VS-4 and isobutyl chloroformate. ESI-TOF-MS m/z 610.3707, calcd for C₃₄H₅₃NO₇Na ([M+Na]⁺) 610.3714.

### EXAMPLE 6

Compounds I wherein R¹ is CO₂H, R² is H or CH₃, R³ is RCNHCO and R⁴ is H or RCNHCO were prepared from ambruticin VS-4 or VS-5 per the following equation:

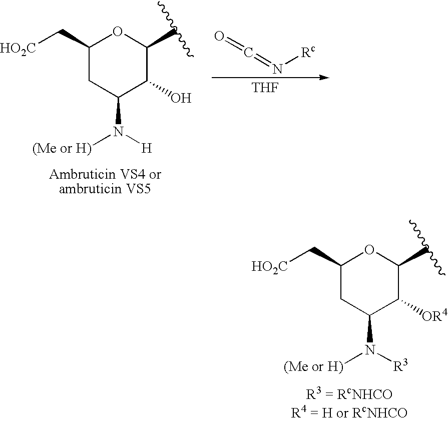

The following general procedure was used: To a suspension of ambruticin VS-4 or ambruticin VS-5 (0.1 mmol) in dry THF (1 mL) was added the isocyanate (0.5 mmol). After the mixture was stirred at 50° C. for 20 h, 300 mg of PS-TsNHNH₂ resin (Argonaut, Calif.) was added, and the mixture was stirred at room temperature overnight. The mixture was then diluted in methanol and filtered to remove the resin. The filtrate was concentrated on a rotary evaporator. The residue was re-dissolved ethyl acetate. The solution was washed with 0.1 M HCl (aq) and brine, and dried over sodium sulfate. The salt was removed by filtration and the filtrate was evaporated to dryness. The crude product was purified by reversed-phase HPLC, eluted using a gradient of acetonitrile in water containing 0.1% acetic acid. The product was obtained as a white solid after lyophilization of desired fractions.

Compound I-q ((5S,6R)-5-(3-allyl-1-methylureido)-6-hydroxypolyangioic acid) was synthesized from ambruticin VS4 and allyl isocyanate. ESI-TOF-MS m/z 593.3582, calcd for C₃₃H₅₀N₂O₆Na ([M+Na]⁺) 593.3561.

Compound I-cc ((5S,6R)-5-(3-allyl-1-methylureido)-6-(allylcarbamoyl)polyangioic acid) was a side product from the preparation of compound I-q, above. ESI-TOF-MS m/z 676.3905, calcd for C₃₇H₅₅N₃O₇Na ([M+Na]⁺) 676.3932.

Compound I-p ((5S,6R)-5-(3-benzyl-1-methylureido)-6-hydroxypolyangioic acid) was synthesized from ambruticin VS-4 and benzyl isocyanate. ESI-TOF-MS nz/z 643.3746, calcd for C₃₇H₅₂N₂O₆Na ([M+Na]⁺) 643.3718.

Compound I-dd ((5S,6R)-5-(3-benzyl-1-methylureido)-6-(benzylcarbamoyl)polyangioic acid) was a side product from synthesis of compound I-p, above. ESI-TOF-MS m/z 776.4254, calcd for C₄₅H₅₉N₃O₇Na ([M+Na]⁺) 776.4245.

Compound I-s ((5S,6R)-5-(3-benzylureido)-6-hydroxypolyangioic acid) was synthesized from ambruticin VS-5 and benzyl isocyanate. ESI-TOF-MS m/z 629.3567, calcd for C₃₆H₅₀N₂O₆Na ([M+Na]⁺) 629.3561.

### EXAMPLE 7

Compounds I wherein R¹ is CH₂OH, R² and R³ are H or CH₃, and R⁴ is H were prepared from ambruticin VS-3, VS-4, or VS-5 or compound I-fff or I-ggg per the following equation:

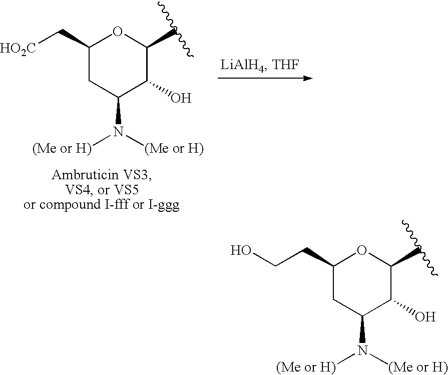

The following general procedure was used: To a suspension of ambruticin VS compound (0.1 mmol) in dry THF (10 mL) was added a solution of 1 M lithium aluminum hydride in THF (1 mL). After the mixture was heated to 50° C. for 1˜4 h, it was cooled in an ice-bath, and a few drops of water was added, followed by magnesium sulfate (25 mg). The precipitate was removed by filtration and thoroughly washed with ethyl acetate. The combined filtrate was evaporated to give an oil, which was purified by reversed-phase HPLC to give the product as a colorless oil.

Compound I-ee ((5S,6R)-5-(methylamino)polyangi-1,6-diol) was synthesized from ambruticin VS-4. ESI-TOF-MS m/z 474.3570, calcd for C₂₉H₄₈NO₄ ([M+H]⁺) 474.3578.

Compound I-ff ((5S,6R)-5-(dimethylamino)polyangi-1,6-diol) was synthesized from ambruticin VS-3. ESI-TOF-MS m/z 488.3737, calcd for C₃₀H₅₀NO₄ ([M+H]⁺) 488.3734.

Compound I-iii ((5S,6R)-5-(2,2,2-trifluroethyl)aminopolyangi-1,6-diol) was synthesized from compound I-fff. ESI-TOF-MS m/z 542.3430, calcd for C₃₀H₄₇F₃NO₄ ([M+H]⁺) 542.2452.

Compound I-jjj ((5S,6R)-5-(2,2-difluroethyl)aminopolyangi-1,6-diol) was synthesized from compound I-ggg. ESI-TOF-MS m/z 524.3557, calcd for C₃₀H₄₈F₂NO₄ ([M+H]⁺) 524.3546.

### EXAMPLE 8

Polyangiamide (ambruticin amide) compounds can be prepared according to the following illustrative procedure for compound I-hh ((5S,6R)-5-(dimethylamino)polyangiamide).

To a solution of ambruticin VS-3 (35 mg) in dry THF (0.1 mL) cooled at 0° C. was added triethylamine (13 μL), followed by ethyl chloroformate (9 μL). After the mixture was stirred at 0° C. for 30 min., a solution of 28% aqueous ammonia (90/L) in THF (0.5 mL) was added. The mixture was allowed to warm to room temperature over 1 h with stirring. Water and ethyl acetate were added. The organic layer was washed with saturated NaCl (aq), dried over anhydrous sodium sulfate, filtered, and evaporated in vacuo. The crude product was purified by reversed-phase HPLC, eluted using a gradient of acetonitrile in water containing 0.1% acetic acid. Compound I-hh was obtained as a white solid (17 mg) after lyophilization of desired fractions. ESI-TOF-MS m/z 501.3691, calcd for C₃₀H₄₉N₂O₄ ([M+H]⁺) 501.3687.

Compound I-ii ((5S,6R)-5-(dimethylamino)-6-ethoxycarbonyloxy-polyangiamide) was obtained as a by-product in the preparation of compound I-hh, isolated as a white solid (5 mg). ESI-TOF-MS m/z 573.3903, calcd for C₃₃H₅₃N₂O₆ ([M+H]⁺) 573.3898.

### EXAMPLE 9

Ambruticin VS compounds having an inverted (5R) stereochemistry at position C5 can be made from ambruticin S. In one approach, ambruticin S is oxidized directly to 5-keto ambruticin S using Dess-Martin periodinane, although it appears that the yield is rather low. Reductive amination followed by separation of epimers affords 5R ambruticin VS compound.

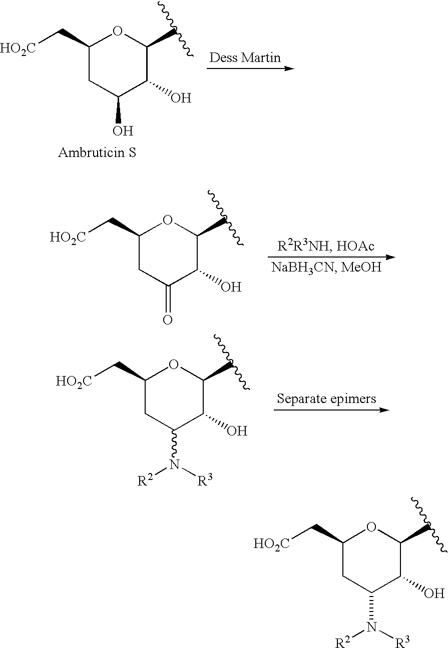

In an alternative approach, which may be preferable, ambruticin S is first converted to the methyl ester and then oxidized to 5-keto ambruticin S methyl ester, as disclosed in Conner et al., U.S. RE 30,339 (1980), who reported difficulties in oxidizing the acid directly. The keto ester is then reductively aminated, the epimers are separated, and the 5R ester is hydrolyzed to afford the 5R ambruticin VS compound.

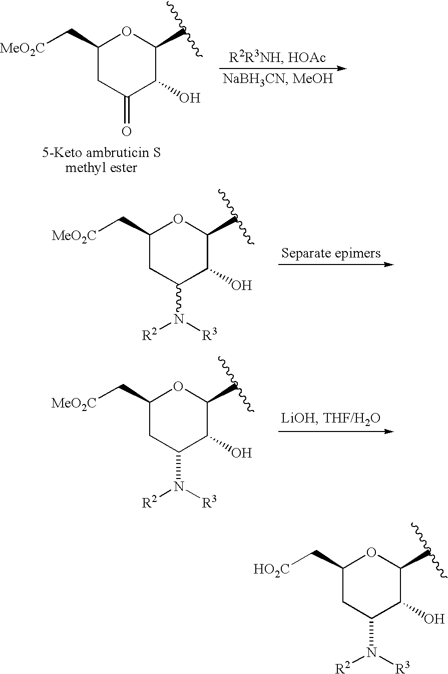

The 5R-ambruticin VS compounds so produced can then be used to make further ambruticin derivatives, by methods analogous to those described in Examples 1 through 8 hereinabove.

### EXAMPLE 10

Compounds I wherein R¹ is CO₂H; R² is H; R³ is alkyl, cycloalkyl, etc.; and R⁴ is H ((5S,6R)-5-(Alkylamino)-6-hydroxypolyangioic acid) were prepared from ambruticin VS-5 per the following series of equations:

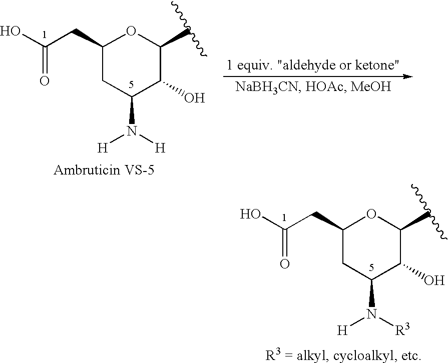

To a solution of ambruticin VS-5 (0.1 mmol) in methanol (1 ml) was added the aldehyde or ketone (0.1 mmol) and acetic acid (0.4 mmol), followed by sodium cyanoborohydride (0.2 mmol). The solution was stirred at 20 to 25° C. until all of the ambruticin VS-5 was consumed. The reaction mixture was concentrated on a rotary evaporator, re-dissolved in a mixture of water-acetonitrile, filtered through a one-gram plug of C-18 silica gel, and purified by reversed-phase HPLC, eluted using a gradient of acetonitrile in water containing 0.1% acetic acid. The product was obtained as a white solid after lyophilization of desired fractions.

Compound I-jj ((5S,6R)-5-(cyclobutylamino)-6-hydroxypolyangioic acid) was synthesized using cyclobutanone. ESI-TOF-MS m/z 528.3686, calcd for C₃₂H₅₀NO₅ ([M+H]⁺) 528.3684.

Compound I-kk ((5S,6R)-5-(isopropylamino)-6-hydroxypolyangioic acid) was using acetone. ESI-TOF-MS m/z 516.3682, calcd for C₃₁H₅₀NO₅ ([M+H]⁺) 516.3684.

Compound I-ll ((5S,6R)-5-[(2-hydroxyethyl)amino]-6-hydroxypolyangioic acid) was synthesized using glycolaldehyde dimer. ESI-TOF-MS m/z 518.3494, calcd for C₃₀H₄₈NO₆ ([M+H]⁺) 518.3476.

Compound I-mm ((5S,6R)-5-(ethylamino)-6-hydroxypolyangioic acid) was synthesized using acetaldehyde. ESI-TOF-MS m/z 502.3533, calcd for C₃₀H₄₈NO₅ ([M+H]⁺) 502.3527.

### EXAMPLE 11

Compounds I wherein R¹ is CH₂OH; R² is CH₃; R³ is alkyl, etc.; and R⁴ is H ((5S,6R)-5-(alkylamino)polyangi-1,6-diol) were prepared from ambruticin VS-4 per the following series of equations:

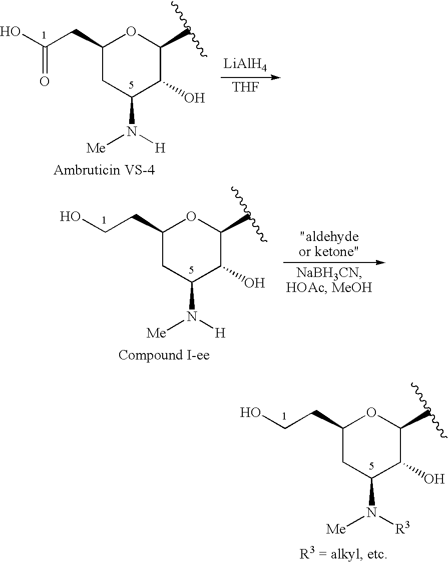

Compound I-ee was prepared using the method of Example 7. To a solution of compound I-ee (0.1 mmol) in methanol (1 mL) was added the aldehyde or ketone (0.2 mmol) and acetic acid (0.4 mmol), followed by sodium cyanoborohydride (0.2 mmol). The solution was stirred at 20 to 25° C. (for reactive aldehydes) or 50 to 60° C. (for less reactive aldehydes and ketones) until all of Compound I-ee was consumed. The reaction mixture was concentrated on a rotary evaporator, re-dissolved in a mixture of water-acetonitrile, filtered through a one-gram plug of C-18 silica gel, and purified by reversed-phase HPLC, eluted using a gradient of acetonitrile in water containing 0.1% acetic acid. The product was obtained as a white solid after lyophilization of desired fractions.

Compound I-nn (5S,6R)-5-[N-(2-hydroxyethyl)methylamino]polyangi-1,6-diol) was synthesized using glycolaldehyde dimer at room temperature. ESI-TOF-MS m/z 518.3839, calcd for C₃₁H₅₂NO₅ ([M+H]⁺) 518.3840.

Compound I-oo ((5S,6R)-5-(N-isoproyl-methylamino)polyangi-1,6-diol) was synthesized using acetone at 50° C. ESI-TOF-MS m/z 516.4038, calcd for C₃₂H₅₄NO₄ ([M+H]⁺) 516.4047.

### EXAMPLE 12

Compounds I wherein R¹ is 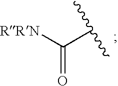
R² is CH₃; R³ is CH₃; and R⁴ is H were prepared from ambruticin VS-3 per the following series of equations:

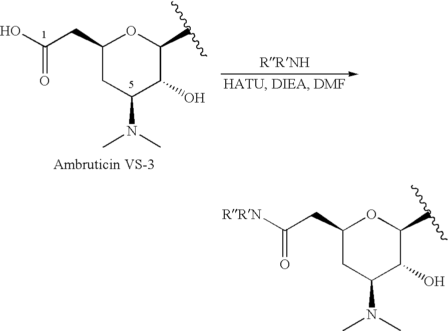

To a solution of ambruticin VS-3 (0.1 mmol) in N,N-dimethylformamide (DMF, 1 mL) was added the amine (0.2 mmol) and N,N-diisopropylethylamine (0.3 mmol), followed by O-(7-azabenzotriazol-1-yl)-N,N,N′,N′-tetramethyluronium hexafluorophosphate (HATU, 0.12 mmol). After being stirred at 20 to 25° C. for 20 h, the reaction mixture was diluted with ethyl acetate (ca. 30 mL). The solution was then washed with saturated aqueous sodium bicarbonate (30 mL), brine (30 mL), and dried over anhydrous sodium sulfate. The salt was removed by filtration and the filtrate was evaporated to dryness. The crude product was purified by reversed-phase HPLC, eluted using a gradient of acetonitrile in water containing 0.1% acetic acid. The product was obtained as a white solid after lyophilization of desired fractions as determined by HPLC/MS.

Compound I-pp (5S,6R)-1-(azetidin-1-yl)-5-dimethylamino-6-hydroxypolyangi-1-one was synthesized using ambruticin VS-3 and azetidine. ESI-TOF-MS m/z 541.3984, calcd for C₃₃H₅₃N₂O₄ ([M+H]⁺) 541.4000.

Compound I-qq (5S,6R)-5-dimethylamino-6-hydroxy-N-[(2-dimethylamino)ethyl]polyangiamide was synthesized using ambruticin VS-3 and N,N-dimethylethylenediamine. ESI-TOF-MS m/z 572.4408, calcd for C₃₄H₅₈N₃O₄ ([M+H]⁺) 572.4422.

Compound I-rr (5S,6R)-5-dimethylamino-6-hydroxy-N-(2-hydroxyethyl)polyangiamide was synthesized using ambruticin VS-3 and ethanolamine. ESI-TOF-MS m/z 545.3938, calcd for C₃₂H₅₃N₂O₅ ([M+H]⁺) 545.3949.

Compound I-ss (5S,6R)-5-dimethylamino-6-hydroxy-N-(methoxycarbonylmethyl)polyangiamide was synthesized using ambruticin VS-3 and methyl glycinate hydrochloride. ESI-TOF-MS nVz 573.3907, calcd for C₃₃H₅₃N₂O₆ ([M+H]⁺) 573.3898.

Compound I-tt (5S,6R)-5-dimethylamino-6-hydroxy-N-methoxy-N-methylpolyangiamide was synthesized using ambruticin VS-3 and N,O-dimethylhydroxylamine. ESI-TOF-MS m/z 545.3928, calcd for C₃₂H₅₃N₂O₅ ([M+H]⁺) 545.3949.

### EXAMPLE 13

Compounds I wherein R¹ is “R^{x}—O˜N═CH—” (R^{x} is H or an alkyl), R² is CH₃ or H; R³ is CH₃; and R⁴ is H were prepared from ambruticin VS-3 per the following series of equations:

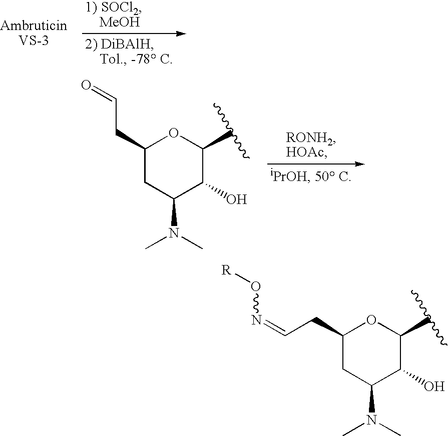

To a solution of ambruticin VS-3 (0.45 g, 0.9 mmol) in methanol (10 mL) cooled in an ice bath was added thionyl chloride (0.08 mL, 1.1 mmol). The mixture was allowed to warm to room temperature with stirring over 4.5 h. The mixture was concentrated on a rotary evaporator, re-dissolved in ethyl acetate. The solution was washed with saturated aqueous sodium bicarbonate, brine, and dried over anhydrous sodium sulfate. The drying agent was removed by filtration. The filtrate was evaporated to dryness in vacuo. Methyl (5S,6R)-5-dimethylamino-6-hydroxypolyangiate (ambruticin VS-3 methyl ester) was obtained as a yellow solid (0.42 g). To a solution of methyl (5S,6R)-5-dimethylamino-6-hydroxypolyangiate (0.35 g, 0.7 mmol) in dry toluene (10 mL) cooled at −78° C. under nitrogen atmosphere was added 1.0 M solution of diisobutylaluminum hydride in toluene (2.5 mL, 2.5 mmol). After the mixture was stirred at −78° C. for 5 minutes, water (0.2 mL) was added, followed by ethyl acetate. The mixture was stirred at room temperature for 20 minutes, then dried with anhydrous sodium sulfate. The drying agent was removed by filtration. The filtrate was evaporated to dryness in vacuo. (5S,6R)-5-Dimethylamino-6-hydroxypolyangial was obtained in quantitative yield as a light yellow solid.

To a solution of (5S,6R)-5-dimethylamino-6-hydroxypolyangial (0.05 mmol) in 2-propanol (0.2 mL) was added the alkoxylamine (0.25 mmol, the hydrochloride salt is neutralized with aq. NaOH before addition if applicable), followed by acetic acid (0.2 mmol). The mixture was heated at 50° C. overnight. The mixture was concentrated in vacuo, re-dissolved in 1:1 water/acetonitrile, filtered, and purified by reversed-phase HPLC on a Varian Metasil Basic column, eluted using a gradient of acetonitrile in water containing 0.1% acetic acid. The product (about 1:1 mixture of E/Z isomers) was obtained as a white solid after lyophilization of desired fractions as determined by HPLC/MS.

Compound I-uu (5S,6R)-5-dimethylamino-6-hydroxypolyangial oxime (R═H) was synthesized using (5S,6R)-5-dimethylamino-6-hydroxypolyangial and hydroxylamine. ESI-TOF-MS m/z 501.3665, calcd for C₃₀H₄₉N₂O₄ ([M+H]⁺) 501.3687.

Compound I-vv (5S,6R)-5-dimethylamino-6-hydroxypolyangial O-methyl oxime (R=Me) was synthesized using (5S,6R)-5-dimethylamino-6-hydroxypolyangial and methoxylamine. ESI-TOF-MS m/z 515.3827, calcd for C₃₁H₅₁N₂O₄ ([M+H]⁺) 515.3843.

Compound I-ww (5S,6R)-5-dimethylamino-6-hydroxypolyangial Ocarboxymethyl oxime (R═CH₂CO₂H) was synthesized using (5S,6R)-5-dimethylamino-6-hydroxypolyangial and carboxymethoxylamine. ESI-TOF-MS m/z 559.3721, calcd for C₃₂H₅₁N₂O₆ ([M+H]⁺) 559.3742.

Compound I-xx (5S,6R)-5-dimethylamino-6-hydroxypolyangial O-tert-butyl oxime (R=^{t}Bu) was synthesized using (5S,6R)-5-dimethylamino-6-hydroxypolyangial and tert-butoxylamine. ESI-TOF-MS m/z 557.4271, calcd for C₃₄H₅₇N₂O₄ ([M+H]⁺) 557.4313.

Compound I-yy (5S,6R)-5-dimethylamino-6-hydroxypolyangial O-4-nitrobenzyl oxime (R=p-NO₂—C₆H₄—CH₂) was synthesized using (5S,6R)-5-dimethylamino-6-hydroxypolyangial and 4-nitrobenzyloxylamine.

Compound I-zz (5S,6R)-5-methylamino-6-hydroxypolyangial oxime was synthesized from ambruticin VS-4 following the following scheme. ESI-TOF-MS m/z 487.3513, calcd for C₂₉H₄₇N₂O₄ ([M+H]⁺) 487.3530.

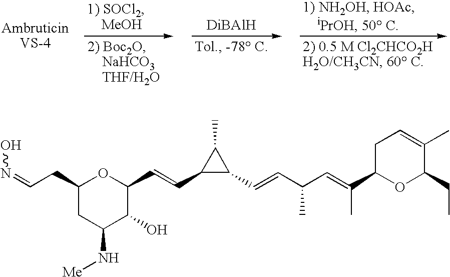

### EXAMPLE 14

Compounds I wherein R¹ is R′R″ NCH₂; R² and R³ are CH₃; and R⁴ is H were prepared from ambruticin VS-3 per the following series of equations:

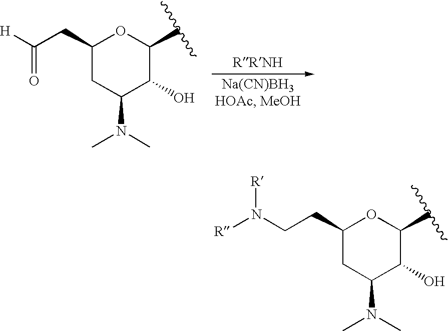

To a solution of (5S,6R)-5-dimethylamino-6-hydroxypolyangial (0.05 mmol) in methanol (0.5 mL) was added the amine (0.25 mmol), acetic acid (0.2 mmol), and sodium cyanoborohydride (0.1 mmol). After stirred at room temperature overnight, the mixture was concentrated in vacuo, re-dissolved in 1:1 water/acetonitrile, filtered, and purified by reversed-phase HPLC, eluted using a gradient of acetonitrile in water containing 0.1% acetic acid. The product was obtained as a white solid after lyophilization of desired fractions as determined by HPLC/MS.

Compound I-aaa (5S,6R)-1-(azetidin-1-yl)-5-(dimethylamino)polyangi-6-ol was synthesized using (5S,6R)-5-dimethylamino-6-hydroxypolyangial and azetidine. ESI-TOF-MS m/z 527.4185, calcd for C₃₃H₅₅N₂O₃ ([M+H]⁺) 527.4207.

Compound I-bbb (5S,6R)-1-amino-5-(dimethylamino)polyangi-6-ol was synthesized using (5S,6R)-5-dimethylamino-6-hydroxypolyangial and ammonium acetate. ESI-TOF-MS m/z 487.3896, calcd for C₃₀H₅₁N₂O₃ ([M+H]⁺) 487.3894.

Compound I-ccc (5S,6R)-1-[(S)-2-carboxypyrrolidin-1-yl]-5-(dimethylamino)polyangi-6-ol was synthesized using (5S,6R)-5-dimethylamino-6-hydroxypolyangial and L-proline. ESI-TOF-MS m/z 585.4234, calcd for C₃₅H₅₇N₂O₅ ([M+H]⁺) 585.4262.

Compound I-ddd (5S,6R)-1-[(R)-2-carboxypyrrolidin-1-yl]-5-(dimethylamino)polyangi-6-ol was synthesized using (5S,6R)-5-dimethylamino-6-hydroxypolyangial and D-proline. ESI-TOF-MS m/z 585.4231, calcd for C₃₅H₅₇N₂O₅ ([M+H]⁺) 585.4262.

Compound I-eee (5S,6R)-1-(azetidin-1-yl)-5-aminopolyangi-6-ol was synthesized using ambruticin VS-5 in the following scheme:

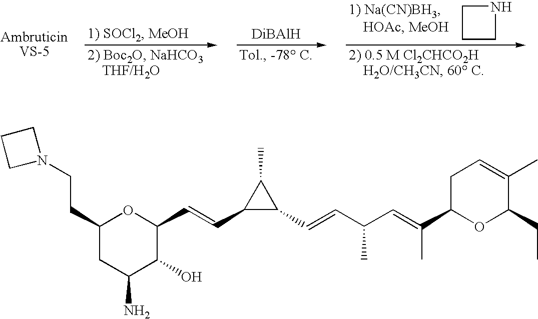

ESI-TOF-MS m/z 499.3878, calcd for C₃₁H₅₁N₂O₃ ([M+H]⁺) 499.3894.

### EXAMPLE 15

A mixture of 5R and 5S isomers (Compound I-kkk; (6R)-5-azetidinyl-6-hydroxypolyangioic acid) was synthesized using the following procedure

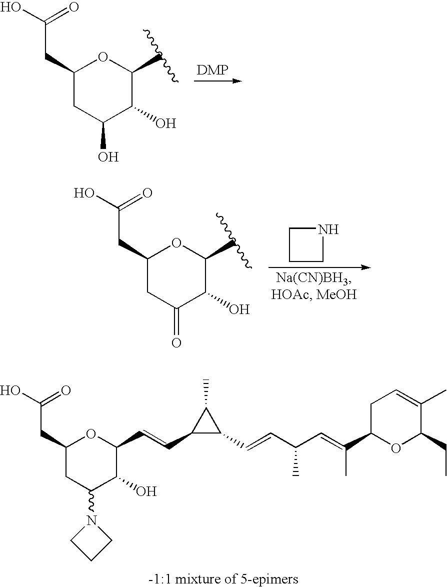

To a solution of ambruticin S (48 mg, 0.1 mmol) in dichloromethane was added Dess-Martin periodinane (85 mg, 0.2 mmol). After the solution was stirred at 20 to 25° C. for 16 h, 0.04 mL of 1 M Na₂S₂O₃ (aq) was added. The mixture was stirred for 20 min. Ethyl acetate was added. The organic phase was separated and washed with saturated aqueous sodium bicarbonate, brine, and dried over anhydrous sodium sulfate. The drying agent was removed by filtration and the filtrate was evaporated to dryness. The crude product was purified by reversed-phase HPLC, eluted using a 30 min-gradient of 25 to 75% acetonitrile in water containing 0.1% acetic acid. (6R)-5-Oxo-6-hydroxypolyangioic acid was obtained as a white solid after lyophilization of desired fractions as determined by HPLC/MS. Yield ˜10%.

To a solution of (6R)-5-oxo-6-hydroxypolyangioic acid (˜10 mg) in methanol was added azetidine (7 μL), acetic acid (10 μL), and sodium cyanoborohydride (6 mg). After stirred at room temperature overnight, the mixture was concentrated in vacuo, re-dissolved in 1:1 water/acetonitrile, filtered, and purified by reversed-phase HPLC on a Varian Metasil Basic column, eluted using a gradient of acetonitrile in water containing 0.1% acetic acid. The product (˜1:1 mixture of epimers) was obtained as a white solid (1.2 mg) after lyophilization of desired fractions as determined by HPLC/MS. ESI-TOF-MS m/z 514.3507, calcd for C₃₁H₄₈NO₅ ([M+H]⁺) 514.3527. One skilled in the art can isolate/separate the 5R and 5S isomers of compound I-kkk using standard procedures in the art.

### EXAMPLE 16

Compound II-b ((5S,6R)-5-dimethylamino-15-desmethylpolyangi-1,6-diol) was synthesized using the following procedure:

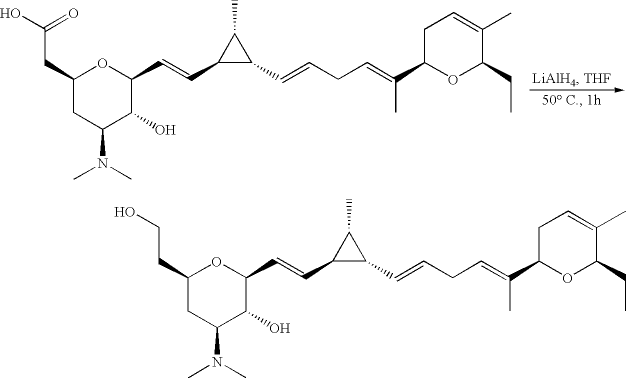

To a suspension of compound II-a ((5S,6R)-5-dimethylamino-6-hydroxy-15-desmethylpolyangioic acid (15-desmethyl ambruticin VS-3)) (8 mg, 0.016 mmol) in dry THF (0.8 mL) was added a solution of 1 M lithium aluminum hydride in THF (0.16 mL). After the mixture was heated at 50° C. under nitrogen atmosphere for 1 h, it was cooled in an ice-bath, and a few drops of water was added, followed by magnesium sulfate (25 mg). The precipitate was removed by filtration and thoroughly washed with THF. The combined THF solutions were evaporated to dryness. The residue was re-dissolved in 1:3 water/acetonitrile and filtered through a 0.2 μm PTFE filter. The filtrate was purified by reversed-phase HPLC on a Varian Metasil Basic column, eluted using a gradient of acetonitrile in water containing 0.1% acetic acid. The product was obtained product as a light yellow gel after lyophilization of desired fractions as determined by HPLC/MS. ESI-TOF-MS m/z 474.3545, calcd for C₂₉H₄₈NO₄ ([M+H]⁺) 474.3578.

### EXAMPLE 17

The following procedure was used to synthesize 16,17-epoxy analogs.

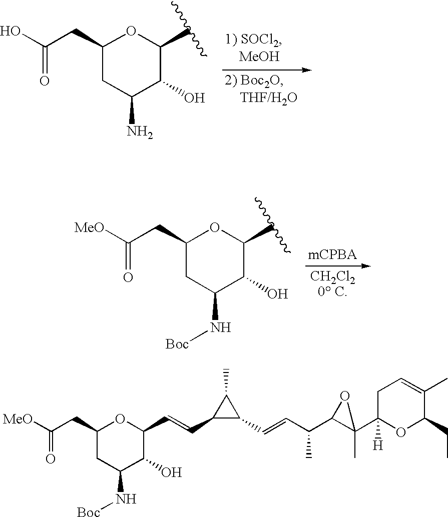

To a solution of ambruticin VS-5 (0.12 g, 0.25 mmol) in methanol (6 mL) was added thionyl chloride (0.04 mL, 0.5 mmol). After the mixture was stirred at −40 to 25° C. overnight, HPLC analysis showed that the reaction was complete. The reaction mixture was stirred with aqueous sodium bicarbonate (20 mL) for 10 minutes. The mixture was concentrated on a rotary evaporator to remove methanol and extracted with ethyl acetate. The combined extracts were washed with water, aqueous sodium bicarbonate, and brine, and dried over anhydrous sodium sulfate. The drying agent was removed by filtration and the filtrate was evaporated to dryness. Methyl (5S,6R)-5-amino-6-hydroxypolyangiate (ambruticin VS-5 methyl ester) was obtained as a yellow solid (0.12 g).

To a solution of methyl (5S,6R)-5-amino-6-hydroxypolyangiate (95 mg, 0.2 mmol) in tetrahydrofuran (6 mL) was added di-tert-butyl dicarbonate (85 mg, 0.4 mmol) at 0° C. The mixture was allowed to warm to 25° C. in 1.5 h, when TLC indicated that the reaction was complete. After the solvent was removed, the crude product was purified by flash chromatography on silica gel eluted with 0-30% ethyl acetate in hexane. Methyl (5S,6R)-5-tert-butoxycarbonylamino-6-hydroxypolyangiate was obtained as a light yellow gel (60 mg). ESI-TOF-MS m/z 610.3696, calcd for C₃₄H₅₃NO₇Na ([M+Na]⁺) 610.3669.

To a solution of methyl (5S,6R)-5-tert-butoxycarbonylamino-6-hydroxypolyangiate (0.3 g, 0.5 mmol) in dichloromethane (12 mL) cooled at −20° C. was added 3-chloroperbenzoic acid (mCPBA) in portions. The reaction was monitored by TLC. Three portions of mCPBA (130 mg, 45 mg, and 45 mg, total 1.25 mmol) were added over a 5 h period to consume most of the starting material. TLC showed two major products and several minor products. The mixture was stirred with aqueous sodium thiosulfate for 20 minutes and was extracted with ethyl acetate. The combined extracts were washed with water, aqueous sodium bicarbonate, and brine, and dried over anhydrous sodium sulfate. The drying agent was removed by filtration and the filtrate was evaporated to dryness. Flash chromatography of the crude product on silica gel column eluted with 0-50% ethyl acetate in hexane gave a mixture of the 16,17-epoxy derivatives, which was purified by reversed-phase HPLC on a Varian Metasil Basic column, eluted using a gradient of acetonitrile in water containing 0.1% acetic acid. Two products, methyl (5S,6R,16R,17R)-5-tert-butoxycarbonylamino-6-hydroxy-16,17-epxoy-16,17-dihydropolyangiate and methyl (5S,6R,16S,17S)-5-tert-butoxycarbonylamino-6-hydroxy-16,17-epxoy-16,17-dihydropolyangiate, were obtained as white solids.

Compound V-a methyl ((5S,6R,16R,17R)-5-tert-butoxycarbonylamino-6-hydroxy-16,17-epxoy-16,17-dihydropolyangiate). ESI-TOF-MS m/z 626.3651, calcd for C₃₄H₅₃NO₈Na ([M+Na]⁺) 626.3663.

Compound V-b methyl ((methyl (5S,6R,16S,17S)-5-tert-butoxycarbonylamino-6-hydroxy-16,17-epxoy-16,17-dihydropolyangiate). ESI-TOF-MS m/z 626.3664, calcd for C₃₄H₅₃NO₈Na ([M+Na]⁺) 626.3663.

Attempts to remove the Boc protecting groups under acidic conditions resulted in rearrangement of the epoxide and/or decomposition. An alternative route was used to obtain the unprotected 16,17-epoxy compounds.

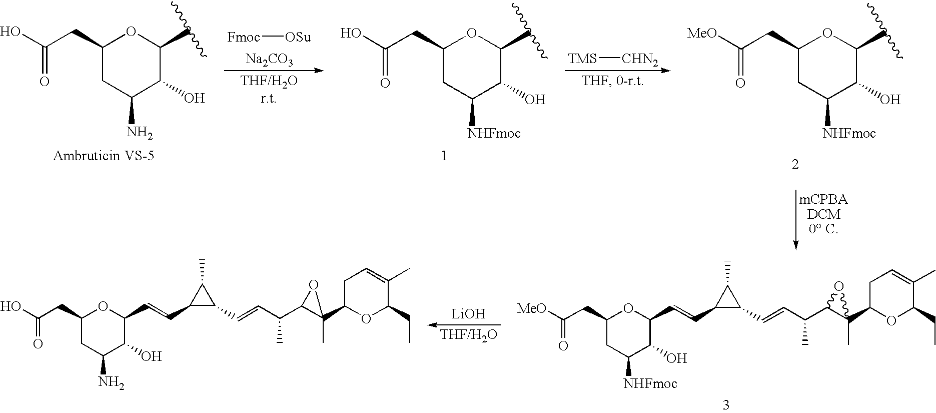

To a mixture of ambruticin VS-5 (0.5 g, 1 mmol) in mixed tetrahydrofuran (6 mL) and 1 M aqueous sodium carbonate (2 mL) was slowly added a solution of Fmoc-OSu (0.5 g, 1.5 mmol) in THF. The mixture was stirred overnight, maintaining the pH at ˜9. The mixture was concentrated to remove tetrahydrofuran and washed with ethyl ether. The aqueous layer (turbid) was acidified with 0.1 M aqueous HCl to pH ˜5 and extracted with ethyl acetate. The combined ethyl acetate extracts were evaporated to dryness. The crude product was purified by flash chromatography on silica gel eluted with 20-30% ethyl acetate in hexane. A solid of 0.13 g of (5S,6R)-5-(9-Fluorenyl)methyloxycarbonylamino-6-hydroxypolyangioic acid (1) was obtained.

To a solution of 1 (0.13 g, 0.19 mmol) in tetrahydrofuran (3 mL) cooled in an ice bath was added dropwisely a 2.0 M solution of (trimethylsilyl)diazomethane in hexanes (1 mL, 2 mmol) under nitrogen atmosphere. The mixture was allowed to warm to room temperature in 2 h. Water was added and the mixture was concentrated to remove tetrahydrofuran. The mixture was extracted with ethyl acetate. The combined ethyl acetate extracts were washed with 0.1 M aqueous HCl, saturated aqueous sodium bicarbonate, and brine, and dried over anhydrous sodium sulfate. The drying agent was removed by filtration and the filtrate was evaporated to dryness. Methyl (5S,6R)-5-(9-fluorenyl)methyloxycarbonylamino-6-hydroxypolyangioate (2) was obtained as a solid (0.11 g).

To a solution of 2 (0.11 g, 0.15 mmol) in dichloromethane (2 mL) cooled at 0° C. was added 3-chloroperbenzoic acid (mCPBA) in three 26 mg portions over 5 h. HPLC analysis showed that most of the starting material had been consumed. The mixture was stirred with aqueous sodium thiosulfate for 20 minutes and was extracted with dichloromethane. The combined extracts were washed with water, aqueous sodium bicarbonate, and brine, and dried over anhydrous sodium sulfate. The drying agent was removed by filtration and the filtrate was evaporated to dryness. Flash chromatography of the crude product on silica gel column eluted with 10-30% ethyl acetate in hexane gave a mixture of the 16,17-epoxy derivatives (3), 13 mg.

To a solution of 3 (13 mg) in tetrahydrofuran (0.4 mL) was added 1 M aqueous lithium hydroxide (0.8 mL). After the mixture was stirred at room temperature for 3 h, it was concentrated on a rotary evaporator to remove tetrahydrofuran. The crude mixture was purified by reversed-phase HPLC on a Varian Metasil Basic column, eluted using a gradient of 25-50% acetonitrile in water containing 0.1% acetic acid. Two products, (5S,6R,16R,17R)-5-amino-6-hydroxy-16,17-epxoy-16,17-dihydropolyangioic acid and (5S,6R,16S,17S)-5-amino-6-hydroxy-16,17-epxoy-16,17-dihydropolyangioic acid, were obtained as white solids (3.7 mg and 2.5 mg).

Compound V-c methyl ((5S,6R,16R,17R)-5-amino-6-hydroxy-16,17-epxoy-16,17-dihydropolyangioic acid). ESI-TOF-MS m/z 512.2964, calcd for C₂₉H₄₃NO₆ ([M+H]⁺) 512.2983.

Compound V-d methyl ((5S,6R,16S,17S)-5-amino-6-hydroxy-16,17-epxoy-16,17-dihydropolyangioic acid). ESI-TOF-MS m/z 512.2958, calcd for C₂₈H₄₃NO₆ ([M+H]⁺) 512.2983.

### EXAMPLE 18

Several 20,21-dihydro analogs were synthesized from compound IV-a using the following procedure:

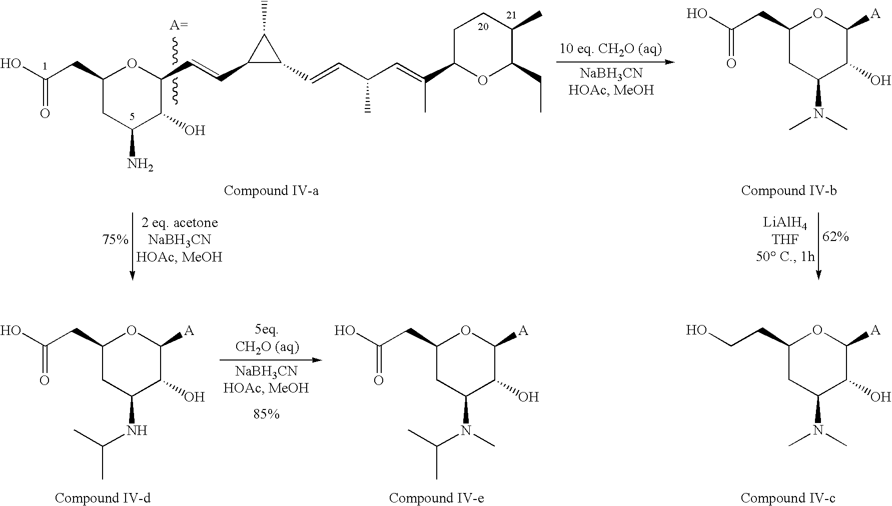

Compound IV-d Compound IV-e Compound IV-c

Compound IV-b was synthesized from compound IV-a using the scheme above. ESI-TOF-MS m/z 504.3676, calcd for C₃₀H₅₀NO₅ ([M+H]⁺) 504.3684.

Compound IV-c was synthesized from compound IV-b using the scheme above. ESI-TOF-MS m/z 490.3885, calcd for C₃₀H₅₂NO₄ ([M+H]⁺) 490.3891.

Compound IV-d was synthesized from compound IV-a using the scheme above. ESI-TOF-MS m/z 518.3838, calcd for C₃₁H₅₂NO₅([M+H]⁺) 518.3840.

Compound IV-e was synthesized from compound IV-d using the scheme above. ESI-TOF-MS m/z 532.3998, calcd for C₃₂H₅₄NO₅ ([M+H]⁺) 532.3997.

Compound IV-b (20,21-Dihydroambruticin VS-3) is also isolated from a side stream in a large scale production of ambruticin VS-3. A mixture obtained from the wild type strain that produces ambruticins VS-3, VS-4, and VS-5 are treated with excess formaldehyde, sodium cyanoborohydride, and acetic acid in methanol to convert all NH₂ and MeNH groups to Me₂N groups. Conversion a 19-g mixture yields ˜14 g of purified ambruticin VS-3 and 280 mg of 20,21-dihydroambruticin VS-3, together with other compounds.

### EXAMPLE 19

C-1 secondary alcohol analogs can be synthesized from ambruticin VS-3 using the following procedure.

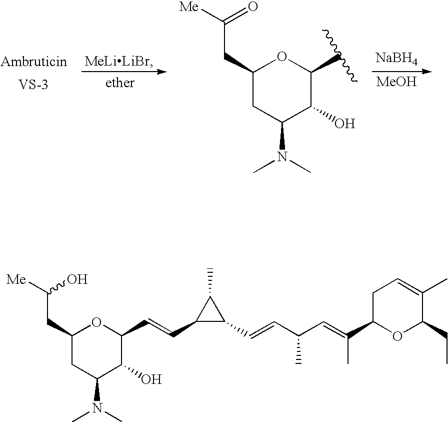

Specifically, compound I-nnn ((5S,6R)-1-methyl-5-(dimethylamino)polyangi-1,6-diol, a mixture of 1R and 1S isomers) was synthesized from ambruticin VS-3 using the following procedure. To a suspension of ambruticin VS-3 (25 mg, 0.05 mmol) in ethyl ether (3 mL) was added 1.5 M methyllithium lithium bromide solution in ether (0.8 mL, 1.2 mmol). After the mixture was stirred at 20° C. for 16 h, it was poured on ice-water. The mixture was extracted with 2×20 mL of ether and 2×20 mL of ethyl acetate. The combined organic solutions were dried over anhydrous magnesium sulfate. The drying agent was removed by filtration. The filtrate was evaporated to dryness in vacuo, giving (5S,6R)-1-methyl-5-dimethylamino-6-hydroxypolyangi-1-one (compound I-mmm) as a colorless gel (25 mg). One skilled in the art can isolate compound 1-mmm using standard methods of the art. To a solution of this product in methanol (2 mL) was added sodium borohydride (19 mg, 0.5 mmol). The reaction was complete as indicated by HPLC analysis after mixture was stirred at 20° C. for 3 h. The mixture was concentrated on a rotary evaporator, re-suspended in 1:3 water/acetonitrile, and filtered through a 0.2 μm PTFE filter. The crude product was purified by reversed-phase HPLC on a Varian Metasil Basic column, eluted using a gradient of acetonitrile in water containing 0.1% acetic acid. The product was obtained as a light yellow gel (15 mg) after lyophilization of desired fractions as determined by HPLC/MS. One skilled in the art can isolate and separate the 1R and 1S isomers using standard methods of the art. ESI-TOF-MS m/z 502.3852, calcd for C₃₁H₅₂NO₄ ([M+H]⁺) 502.3891.

### EXAMPLE 20

C-1 tertiary alcohol analogs can be synthesized from ambruticin VS-3 using the following procedure.

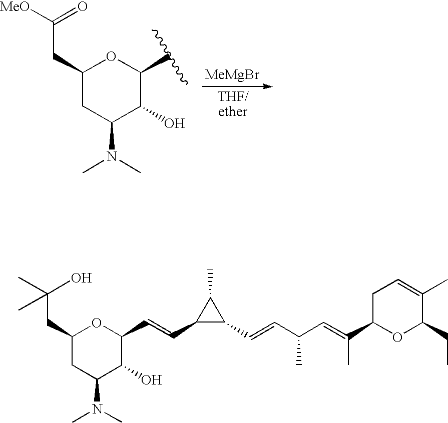

Specifically, compound I-ooo ((5S,6R)-1,1-dimethyl-5-(dimethylamino)polyangi-1,6-diol) was synthesized from ambruticin VS-3 methyl ester using the following procedure.

To a solution of ambruticin VS-3 methyl ester (52 mg, 0.1 mmol) in THF was added 3 M methyl magesium bromide in ether (0.2 ml, 0.6 mmol). The mixture was stirred at room temperature for 30 min and concentrated on a rotary evaporator to dryness. The crude mixture was re-dissolved in 1:1 water/acetonitrile, filtered, and purified by reversed-phase HPLC on a Varian Metasil Basic column, eluted using a gradient of acetonitrile in water containing 0.1% acetic acid. The product (compound I-ooo) was obtained as a white solid (15 mg) after lyophilization of desired fractions as determined by HPLC. ESI-TOF-MS m/z 516.4044, calcd for C₃₂H₅₄NO₄ ([M+H]⁺) 516.4047.

### EXAMPLE 21

Compound I-lll ((5S,6R)-2-ethoxycarbonylamino-5-(dimethylamino)-1-norpolyangi-6-ol) can be synthesized from ambruticin VS-3 using the following procedure.

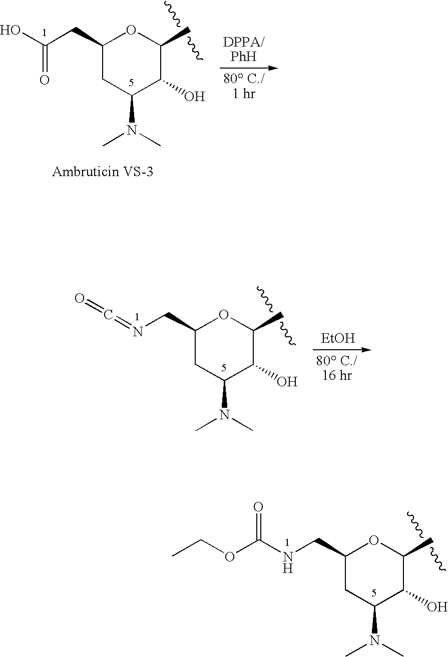

To a solution of ambruticin VS-3 (0.15 mmol) in benzene (5 mL) was added diphenylphosphoryl azide (0.17 mmol) and triethylamine (0.17 mmol). The solution was stirred at 80° C. for 1 h, then was added ethanol (3 mmol) to be stired at 80° C. for 16 h. The reaction mixture was concentrated on a rotary evaporator, re-dissolved in a mixture of water-acetonitrile, filtered through a one-gram plug of C-18 silica gel, and purified by reversed-phase HPLC, eluted using a gradient of acetonitrile in water containing 0.1% acetic acid. The product (compound I-lll) was obtained as a white solid after lyophilization of desired fractions. ESI-TOF-MS m/z 545.3933, calcd for C₃₂H₅₃N₂O₅ ([M+H]⁺) 545.3949.

### EXAMPLE 22

The following describes the construction of the ambS, ambO, ambP and ambM mutants in Sorangium cellulosum So ce10 and the analysis of ambruticin compounds produced.

The construction of the ambS, ambO, ambP and ambM mutants in Sorangium cellulosum So ce10 was peformed using the following method. The nucleotide sequence of the ambO, ambP, and ambM genes are disclosed in U.S. patent application Ser. No. 11/075,185, and WO 2005/086907 (each incorporated herein by reference). Gene regions were amplified by PCR and each amplicon was cloned into the EcoRV site of pKOS175-178, a plasmid that carries the oriT of R⁶K for conjugal transfer and the phleomycin resistance marker for selection in Sorangium. Primer sequences and plasmid names were as follows: ambM, TGATACAACGACGCTTACACG (SEQ ID NO:1) and CTAGCGGAACGACATGGTGAA (SEQ ID NO:2) to give pKOS546-28M; ambS, TAGGCCAGGTTGAGCCATGAG (SEQ ID NO:3) and CTATTGCTCTCTGGCCAGGAG (SEQ ID NO:4) to give pKOS375-155; ambO, TGAGCGGTCGGCGCCAGCTGG (SEQ ID NO:5) and TCACGTGAAGCGCGCCGCGTC (SEQ ID NO:6) to give pKOS375-189O; ambP, TGACACCCGGTACTCCTCAGC (SEQ ID NO:7) and TCAGCGCTTGTCCGCCAGACG (SEQ ID NO:8) to give pKOS375-189P. Each resulting plasmid was introduced by transformation into E. coli strain C-2420 containing the helper conjugative plasmid pKOS1111-47 (Julien et al., 2003). Development of a mariner-based transposon for use in Sorangium cellulosum. Appl Environ Microbiol 69, 6299-6301). The procedure for conjugation of the plasmid from E. coli to So ce10 was previously described (Jaoua et al. (1992). Transfer of mobilizable plasmids to Sorangium cellulosum and evidence for their integration into the chromosome. Plasmid 28, 157-165) and transconjugants were selected on S42 agar containing kanamycin (50 μg/ml) and phleomycin (50 μg/ml) to give strains K546-40M2, K375-167.4, K546-3202, and K546-5P3, respectively. The Sorangium cellulosum cells were maintained using the method of Hofle et al. (1991, Liebigs Ann Chem 1991, 941-945) and Gerth et al. (1996, J Antibiot (Tokyo) 49, 71-75.

The analyses of ambruticin compounds produced by the various mutant strains were performed using the following method. Colonies were inoculated into seed medium (10 g/L maltodextrin, 4 g/L nonfat dry milk, 4 g/L soy peptone, 4 ml/L glycerol, 1 g/L CaCl₂.2H₂O, 1 g/L MgSO₄.7H₂O, 15 mg/L FeCl₃-6H₂O, 25 mM HEPES, pH 7.6) and grown for 2-3 days at 32° C. Production medium (10 g/L maltodextrin, 5 g/L Pharmamedia, 4 g/L nonfat dry milk, 4 g/L soy peptone, 4 ml/L glycerol, 1 g/L CaCl₂.2H₂O, 1 g/L MgSO₄.7H₂O, 120 mg/L FeCl₃-6H₂O, 50 mM HEPES, pH 7.6) containing 40 g/L XAD1180 was inoculated with 10% seed culture and incubated at 32° C. for 8 days. After washing the XAD resin twice with water, the ambruticin compounds were eluted with a volume of methanol equal to half the original culture volume.

To resolve ambruticins containing an amino group, they were chromatographed using the VS method: Agilent Nucleosil C18 column (4×125 mm;), isocratic, 78% methanol, 10 mM ammonium acetate, pH 8.2, 1 ml.min⁻¹, detection at 220 nm. The VS compounds were quantitated from the area under the peak compared to purified standards. Ambruticin S and other ambruticins not containing an amino group were quantitated by the S method: Agilent Eclipse XDB-C8 column (4.6×150 mm), isocratic, 64% acetonitrile, 0.1% acetic acid, 1 ml.min⁻¹, detection at 220 nm. For LC/MS analysis the separation method used a MetaChem Inertsil ODS-3 column (4×100 mm) with a gradient from 30% to 100% acetonitrile in 0.1% acetic acid at 1 ml.min⁻¹ on an Agilent 1100 system with a diode array detector connected to a Perseptive Biosystems Mariner biospectrometry workstation.

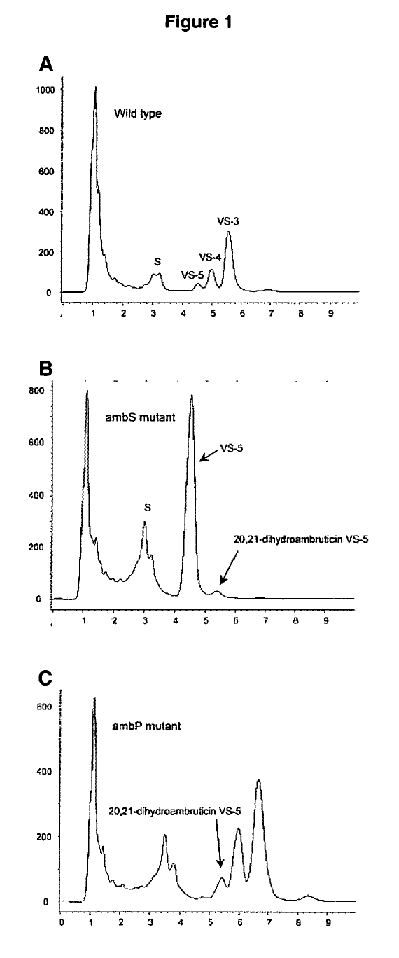

Analysis of the ambS mutant. Disruption of ambS, which encodes a methyl transferase homologue in the ambruticin cluster, gave a strain that no longer produced the N-methylated ambruticins VS-4, VS-3 or VS-1. FIG. 1 shows HPLC-UV analysis of extracts from the ambS⁻ mutant and the wild type strain. The extracts were also analyzed by LC-MS, which verified the absence of the VS-4, VS-3 and VS-1 compounds, and indicated that the small peak eluting after VS-5 had the mass of VS-5 plus two hydrogens. The amount of ambruticin VS-5 produced by the mutant was approximately equal to the sum of ambruticins VS-1, VS-3, VS-4 and VS-5 produced by the wild type strain under the same conditions, indicating that the AmbS protein catalyzes, as the final steps in the pathway, sequential N-methylations of VS-5 to give VS-4, VS-3 and VS-1. Ambruticin VS-5 and the compound two hydrogen atoms heavier were purified from a large scale fermentation of the ambS⁻ strain and the heavier compound was shown by NMR analysis to be 20,21-dihydroambruticin VS-5. Upon careful inspection of LC-MS data from cultures of the wild-type strain, very small amounts of all the 20,21-dihydroambruticins could be detected.

Analysis of the ambO and ambP mutant. The ambruticin gene clusters has a pair of adjacent genes in the same operon encoding a flavin monooxygenase (ambO) and a Rieske iron-sulfur cluster protein (ambP). When either the ambP or ambO genes of Sorangium cellulosum So ce10 was disrupted, the resulting strains produced a set of ambruticins at similar levels to those produced by the wild type strain, except that each eluted later from the reverse-phase HPLC column and had a mass two hydrogen atoms heavier. FIG. 1 compares the HPLC-UV chromatograms of extracts from the ambP⁻ mutant with the wild type strain. The data for the ambP⁻ mutant was identical to that for the ambP⁻ mutant. LC-MS analysis verified that compounds having the mass of each of the ambruticins produced by the wild type were not detected in either mutant. The putative 20,21-dihydroambruticin VS-5 peak co-eluted with a purified standard of this compound. Disruption of either ambP or ambO prevents formation of the 20,21-double bond. The relative level (and estimated absolute level) of each 20,21-dihydroambruticin was similar to that of each corresponding ambruticin produced by the wild type.

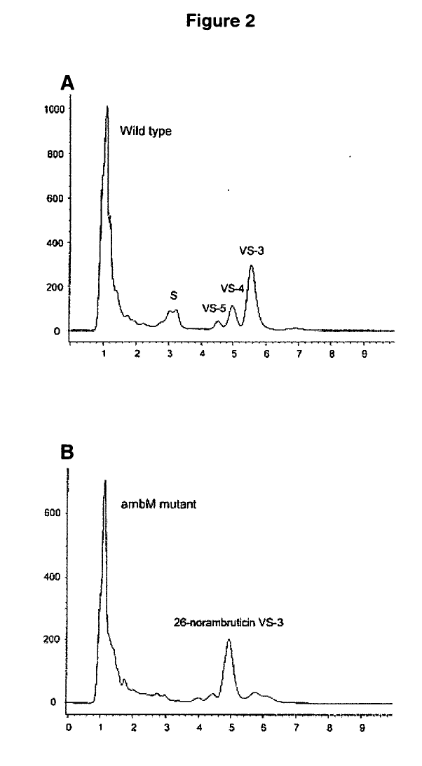

Analysis of the ambM mutant. An ambM mutant was constructed using the same procedure described above. The extract from four 500 ml cultures of K546-40M2 was adjusted to 50% methanol, 50 mM ammonium acetate and loaded onto a 2.5×28 cm column of BakerBond C18. After washing with 50% methanol, 50 mM ammonium acetate, fractions were collected during elution with 80% methanol, 50 mM ammonium acetate at 6 ml/min. Fractions containing 27-norambruticin VS-3 were identified using the analytical HPLC method described above, pooled, and the solvent was exchanged over a 0.5×26 cm BakerBond C18 column to remove the ammonium acetate. The material was dried, dissolved in CD₃OD and analyzed on a Bruker 400 MHz instrument. FIG. 2 shows the HPLC-UV chromatograms of the extracts from the ambM mutant compared to the wild type strain showed a single main peak for the ambM mutant. LC-MS analysis of the extracts indicated that each compound eluting earlier than each of the known ambruticins produced by the wild type had a mass that was 14 atomic mass units lighter, consistent with the loss of a methyl group. The most prevalent compound from the ambM mutant was purified and NMR spectroscopic analysis showed that it is 27-norambruticin VS-3 (compound III-a) having the following structure:

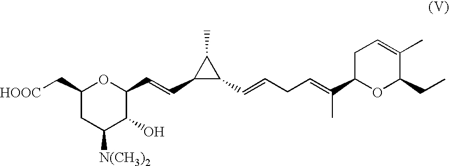

The AmbM protein is a C-methyltransferase, and the ambM mutant produces the set of ambruticins corresponding to those produced by the wild type strain, except that each is missing the C27 methyl group (for example, 27-norambruticin VS-3, 27-norambruticin VS-4, and 27-norambruticin VS-5).

### EXAMPLE 23

The following is a method for constructing a strain of Sorangium cellulosum So ce10 that produces compound III-a (24-norambruticin VS-3).

Replacement of the ambruticin loading AT with the one from module 7. The nucleotide sequence of the ambruticin loading AT and the AT of module 7 of the ambruticin PKS gene cluster is disclosed in U.S. patent application Ser. No. 11/075,185, and WO 2005/086907. Plasmid pKOS396-185A was constructed in several steps. To engineer the AT from module 7 into the loading module, 2 PCR fragments were generated to produce the right and left boundaries of the swap; the left contains the KS-AT boundary and the right contains the AT-ACP boundary. The right fragment was amplified using plasmid pKOS344-112E and the oligo pair, 5′-TTTTAATTAAGAGGAGCATATGGATCCGCAGC (SEQ ID NO: 9) (PacI restriction sites underlined) and 5′-GCCCGCGGCGGTTCCGGGGCCTCCTCGGACACCACATGC (SEQ ID NO: 10). The left fragment was amplified using the same plasmid and oligo pair, 5′-GCCATGTGGTGCTCGAGGAGGCCCCGGAACCGCCGCGGGC (SEQ ID NO: 11) and 5′-TITCTAGACCTAGGGCCATTGAGCGCCG (SEQ ID NO: 12) (AvrII restriction site underlined). The PCR products from these two PCR reactions were joined together using the two products as the template and the two oligos containing the PacI and AvrII restriction sites as primers. The left fragment was amplified in an identical manner. First, two PCR reactions were performed using plasmid pKOS344-112E and the oligo pair, 5′-AATGGCCCTAGGCAGACCGTCGTCAG (SEQ ID NO: 13) (AvrII restriction sites underlined) and 5′-TAGCGCTGGCGCTGGAATGCGTAGGTCGGCAGCTCCACCC (SEQ ID NO: 14); and the other reaction with the oligo pair, 5′-GGGTGGAGCTGCCGACCTACGCATrCCAGCGCCAGCGCTA (SEQ ID NO: 15) and 5′-TTTCTAGAGATCTAGACGAGCGCATCGATG (SEQ ID NO: 16) (BglII restriction site underlined) and they were joined together using the products as templates and the oligos with the restriction sites as primers. The final PCR products were ligated into pCRscript vector (Stratagene) and the DNA sequence was confirmed. The right fragment cleaved with PacI-AvrII and the left fragment with AvrII-BglII were ligated to pKOS249-51 cleaved with PacI-BglII to give pKOS396-185A. This plasmid contains the oriT, which is required for conjugative transfer from E. coli to S. cellulosum. It also contains the Mx9 integrase gene and attP site for site specific recombination in M. xanthus but appears not to function in So ce10, likely due to an inadequate Mx9 attB site in the chromosome. Besides having the engineered AT, pKOS396-185A also contains truncation in the 5′ region of ambA and has the promoter for the epothilone biosynthetic gene positioned just upstream of the engineered ambA.

Integration of pKOS396-185A into So ce10. Transformation of Sorangium cellulosum So ce10 with pKOS396-185A was performed as described using E. coli donor cells C2420 containing the helper plasmid pKOS 111-47⁵. Selection was done on S42 medium containing hygromycin (60 μg/ml) and kanamycin (50 μg/ml).

Production of compound III-a (24-norambruticin VS-3). Ten independent isolates were tested for production. Seed cultures were grown in 25 ml of C307 (per liter 10 g potato starch-soluble (Sigma), 1 g glucose, 5 g select Soytone (Difco), 2 g yeast extract (Fisher), 1 g MgSO₄.7H₂O, 1 g CaCl₂-2H₂O and 0.008 g Fe citrate) in unbaffled 250 ml flasks for two days at 32° C. A 10% v/v inoculum was diluted into 50 ml production media (per liter 5 g maltodextrin DE18 (Cerestar), 2.5 g soy peptone (Marcor), 0.5 g MgSO₄.7H₂O, 0.25 g K₂HPO₄, 50 mM HEPES pH 7.6, 1 g ferric citrate and 10 g XAD 1180) seven days at 32° C. After the fermentation, products were eluted from the XAD using 5 ml methanol.

The loading module was targeted for engineering to make compound III-a (24-norambruticin VS-3). To alter this AT, ambruticin modules containing malonate specific ATs were examined for similarities in reductive domains to those found in the loading module. The most similar was module 7. The following shows the boundaries at the amino and carboxy terminal of the 2 ATs.

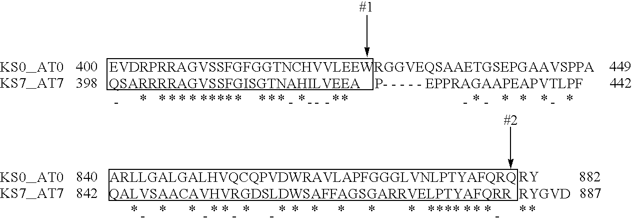

Integration of pKOS396-185A into So ce10. Transformation of Sorangium cellulosum So ce10 with pKOS396-185A was performed as described using E. coli donor cells C2420 containing the helper plasmid pKOS111-47. Selection was done on S42 medium containing hygromycin (60 μg/ml) and kanamycin (50 μg/ml).

Alignment of the boundaries between the KS and AT domains of modules 0 and 7 from the ambruticin PKS. The top box shows the KS domain and the bottom box shows the AT domain. The arrows show the boundaries chosen for AT swaps: #1 is between KS and AT domains, and #2 is at the end of the ATs.

Plasmid pKOS396-185A contains the malonate specific AT from module 7 engineered into the loading module. The plasmid was integrated by homologous recombination that creates an inactive native ambA that allows for expression of the downstream ambruticin genes and expresses the engineered ambA.

Plasmid pKOS396-184A was conjugated into So ce10 and several hygromycin resistant conjugants were obtained. Flask fermentations were performed in the presence of the XAD-1180. Analysis of the eluted material confirmed the production of compound III-a (24-norambruticin VS-3) by LC-MS analysis. This was further confirmed by NMR after production and purification of enough material.

A method for producing and purifying compound II-a is as follows: Seed cultures were inoculated from cells spread on S42 plates containing 200 mg/L hygromycin. A 25-mL tube with five mL of CF₉ medium (Fructose 6 g/L, Casitone (Difco) 9 g/L, MgSO₄.7H₂O g/L, CaCl₂.2H₂O 0.5 g/L, and HEPES (1.0 M, pH 7.6, KOH) 25 mL/L) containing hygromycin (200 μg/ml) was inoculated with a 1 cm2 patch from S42 plates. A ten percent inoculum was used to expand the seed into a 250-mL unbaffled Erlenmeyer flask containing 50 mL of CF₉ medium with hygromycin. The flasks were incubated at 32° C. and 190 rpm on a 2-inch throw shaker for three days. The secondary seed culture was transferred (10% v/v) into a 2.8-L unbaffled Fembach flask containing 500 mL of CF₉—H medium. The Fembach flasks were incubated at 32° C. and 190 rpm on a 2-inch throw shaker for three days. The cultures at all seed stages grew as dispersed cultures.

Production Culture. Two seed cultures were prepared as described above. Each 1-L seed culture was inoculated into a 20-L BiofloIV bioreactor containing 11.0 L of production medium SF-1P (Fructose g/L, Soy Peptone (Marcor) 3 g/L, MgSO₄.7H₂O 1 g/L, CaCl₂.2H₂O 1 g/L, FeCl₃.6H₂O (14.6 g/L in 10 mL/L conc. H₂SO₄) 8 mL/L, XAD-4 20 g/L). The pH of the fermentation was maintained at 7.1 with 2.5 N KOH or 2.5 N H₂SO₄. Airflow was set at 4 L/min, agitation rate at 100 rpm, and overhead pressure at 3 psi. Dissolved oxygen was controlled at 40%. Temperature was controlled at 32.0° C. Cognis Clerol FBA 5059 antifoam was added to prevent foam formation as needed. The culture was fed 3.0 g/L/D fructose and 1.5 g/L/D soytone starting at 48 hours after inoculation and continuing until the end of the fermentation.

Isolation. The XAD-1180 resin (200 mL) was removed from the whole broth by sedimentation. The resin was packed in a glass column (4.5 cm diameter, 28 cm long) and washed with 10 column volumes of water, then with five column volumes of 40% (v/v) methanol:water. The XAD-1180 column was eluted with five column volumes of 100% methanol. The methanol concentration of the eluted fraction was adjusted to 40% (v/v) with water. This solution was loaded at 15 mL/min onto a preconditioned C18 column (2.5 cm diameter, 20 cm long), washed with three column volumes of 40% (v/v) methanol:water, and eluted with 78% (v/v) methanol:50 mM ammonium acetate buffer pH 8.2. Fractions (25 mL) were collected and analyzed by mass spectrometry, and those containing the desired compound were combined. Fractions 7-17 from the C18 chromatography were pooled (275 mL). The methanol concentration of the pooled fractions was adjusted to 40% (v/v) with water and loaded onto a preconditioned HP20SS column (2.5 cm diameter, 20 cm long). The column was washed with two column volumes of 40% (v/v) methanol, then 10 column volumes of water. The product was eluted with 100% methanol (200 mL). The material was then dried resulting in 7 mg of a yellow solid.

Final purification was performed by preparative HPLC using a Metachem Polaris column (2.12 cm diameter, 25 cm long) eluted with 78% (v/v) methanol:50 mM ammonium acetate buffer pH 8.2. Fractions (10 mL) were collected and analyzed by LC/MS. A final desalting step was performed using a preconditioned HP20SS column (1 cm diameter, 3 cm long). The methanol concentration of the pooled fractions (11-14) from the preparative HPLC was adjusted to 40% (v/v) with water and loaded onto the HP20SS column. The column was washed with 10 column volumes of water and eluted with 10 column volumes of methanol. The product-containing eluate was dried yielding 4.7 mg of solids with a purity of 96% (Figure II.20).

24-norambruticin VS-3 analysis. Samples (50 mL) were taken from the bioreactors using 50-mL conical tubes. Medium in the tube was decanted and The resin was stored at −20° C. To elute the compounds, the resin was first washed with 50 mL of water, and then five mL of methanol were added. The XAD extracts were assayed in a Hewlett Packard 1090 HPLC with UV detection at 210 nm. Twenty-five microliters of the supernatant were injected across a 4.6×150 mm, 3.5 Nucleosil column (Agilent) with 78:22 (v/v) methanol:50 mM ammonium acetate, pH 8.2 as the solvent. Under these conditions 24-norambruticin VS-3 was detected at 3.9 min. Compound III-a (24-norambruticin VS-3) was successfully isolated to purity greater than 95%. The isolation involved C18 chromatography followed by HP20SS chromatography then preparative HPLC. Preparative HPLC was needed to separate impurities that could not be resolved with low-pressure chromatography. The overall yield of the purification was 57%. Alternately, the HP20SS chromatography can be performed before the C18, thereby eliminating the need for the C18 step.

### EXAMPLE 24

The antifungal activities of compounds of this invention against two different Aspergillus species were determined by microdilution methods according to the NCCLS reference method no. M38-A, “Reference Method for Broth Dilution Antifungal Susceptibility Testing of Filamentous Fungi; Approved Standard”. Results are presented in Table H and include comparative data for ambruticin S, ambruticin VS-3, ambruticin VS-4, ambruticin VS-5,5-keto ambruticin S, and the structurally unrelated fungicides amphotericin B and itraconazole. MIC₀ indicates the minimal concentration observed to inhibit>95% growth of the fungal species tested relative to the untreated control. All of the values reported are the average of at least two assays (except for I-l and I-x).

TABLE H
Antifungal Activity

MIC₀ (μg/mL)

| | A. flavus ATCC | A. fumigatus ATCC |
|---|---|---|
| Compound | 204304 | 204305 |
| Amphotericin B | 1.67 | 1.53 |
| Itraconazole | 0.47 | 0.58 |
| Ambruticin S | 28.44 | 29.33 |
| Ambruticin VS-3 | 0.64 | 0.44 |
| Ambruticin VS-4 | 0.47 | 0.60 |
| Ambruticin VS-5 | 1.09 | 1.64 |
| 5-keto ambruticin S | 1.0 | 2.0 |
| I-a | 2.0 | 2.0 |
| I-b | 1.5 | 1.5 |
| I-c | 2.0 | 2.67 |
| I-d | 8.0 | 8.0 |
| I-e | 3.33 | 4.0 |
| I-f | 4.67 | 8.0 |
| I-g | 24.0 | 22.67 |
| I-h | 1.0 | 2.1 |
| I-i | >32 | 32 |
| I-j | >32 | 32 |
| I-k | >32 | >32 |
| I-l | 16 | 8 |
| I-m | >32 | >32 |
| I-n | 2 | 1.4 |
| I-o | 32 | 16 |
| I-p | 32 | 32 |
| I-q | 32 | 16 |
| I-r | >32 | >32 |
| I-s | 32 | 16 |
| I-t | 2.25 | 2.25 |
| I-u | 4.5 | 4.25 |
| I-v | 1.1 | 1.32 |
| I-w | 4 | 4.25 |
| I-x | 4 | 2 |
| I-y | 9 | 8.5 |
| I-z | 2.2 | 2.1 |
| I-bb | 32 | 16 |
| I-cc | >32 | >32 |
| I-dd | >32 | >32 |
| I-ee | 3 | 9.5 |
| I-ff | 1.76 | 2.07 |
| I-gg | >32 | >32 |
| I-hh | 2 | 1 |
| I-ii | 1 | 1 |
| I-jj | 5.33 | 6.67 |
| I-kk | 1.1 | 1 |
| I-ll | 10 | 10 |
| I-mm | 0.93 | 1.28 |
| I-nn | 4 | 32 |
| I-oo | 2 | 32 |
| I-pp | 2 | 4 |
| I-qq | >32 | 32 |
| I-rr | 2 | 4 |
| I-ss | 8 | 4 |
| I-tt | 4 | 8 |
| I-zz | 8 | 12 |
| I-fff | 2 | 3 |
| I-ggg | 8 | 4 |
| I-hhh | >32 | >32 |
| I-iii | 16 | 16 |
| I-jjj | 8 | 8 |
| II-a | 0.92 | 0.6 |
| II-b | 8 | 8 |
| III-a | 1.6 | 0.71 |
| IV-a | 0.18 | 0.85 |
| IV-b | 0.44 | 0.88 |
| IV-c | 2 | 2 |
| IV-d | 0.88 | 2.5 |
| IV-e | 1 | 2.5 |
| V-a | >32 | >32 |
| V-b | >32 | >32 |
| V-c | 16 | 8 |
| V-d | >32 | >32 |

### EXAMPLE 25

The compounds of this invention were tested against 3 different strains of Blastomyces dermatitidis and Histoplasma capsulatum. The antifungal activity of the compounds tested were determined by microdilution methods according to the NCCLS reference method no. M38-A, “Reference Method for Broth Dilution Antifungal Susceptibility Testing of Filamentous Fungi; Approved Standard”. Results are presented in Tables I-L. MIC₀ indicates the minimal concentration observed to inhibit>95% growth of the fungal species tested relative to the untreated control. MIC indicates the minimal concentration observed to inhibit 50% growth of the fungal species tested relative to the untreated control. MIC2 indicates the minimal concentration observed to inhibit 80% growth of the fungal species tested relative to the untreated control.

TABLE I
Antifungal Activity

| | MIC₀ (μg/mL) | | | | | | | | | | | |
|---|---|---|---|---|---|---|---|---|---|---|---|---|
| | Blastomyces dermatitidis | | | | | | | | | | | |
| | 04- | 04- | 04- | 05- | 05- | 05- | 05- | 05- | 05- | 05- | R- | R- |
| Compound | 2167 | 2508 | 2915 | 106 | 186 | 1391 | 1707 | 1820 | 1829 | 2020 | 3529 | 35629 |
| Itraconazole | 0.03 | 0.03 | 0.06 | 0.06 | 0.06 | 0.03 | ≦0.015 | ≦0.015 | 0.06 | ≦0.015 | ≦0.015 | ≦0.015 |
| Ambruticin S | 0.125 | 0.25 | 1.0 | 0.5 | 0.125 | 0.06 | ≦0.015 | 0.125 | 0.25 | 0.06 | 0.06 | 0.125 |
| I-v | 0.06 | 0.06 | 0.125 | 0.03 | 0.06 | 0.03 | ≦0.015 | 0.06 | 0.06 | 0.06 | 0.063 | 0.03 |
| I-ff | 0.06 | 0.06 | 0.06 | 0.06 | 0.06 | 0.06 | ≦0.015 | 0.03 | 0.06 | 0.06 | 0.03 | 0.06 |

TABLE J
Antifungal Activity

| | MIC2 (μg/mL) | | |
|---|---|---|---|
| | B. dermatitidis | B. dermatitidis | B. dermatitidis |
| Compound | 05-106 | 05-186 | 05-1291 |
| Ambruticin VS-4 | ≦0.15 | ≦0.15 | ≦0.15 |
| IV-a | ≦0.15 | ≦0.15 | ≦0.15 |

TABLE K
Antifungal Activity

| MIC (μg/mL) | |
|---|---|
| B. dermatitidis | H. capsulatum |

| Compound | Kr | Gu | V | G217B | GS20 |
|---|---|---|---|---|---|
| Amphotericin B | 1 | 2 | 1 | 0.19 | 0.19 |
| I-n | >12.5 | 6.25 | >12.5 | <0.19 | <0.19 |
| I-d | >12.5 | >12.5 | >12.5 | <0.19 | <0.19 |
| I-u | >12.5 | >12.5 | >12.5 | <0.19 | <0.19 |
| I-z | >12.5 | >12.5 | >12.5 | <0.19 | <0.19 |
| I-oo | >12.5 | 0.39 | >12.5 | <0.19 | <0.19 |
| I-v | >12.5 | 12.5 | 12.5 | <0.19 | <0.19 |
| I-y | >12.5 | 3.13 | >12.5 | <0.19 | <0.19 |
| I-t | >12.5 | >12.5 | >12.5 | <0.19 | <0.19 |
| I-kk | >12.5 | 12.5 | >12.5 | <0.19 | <0.19 |
| I-ff | >12.5 | >12.5 | >12.5 | <0.19 | <0.19 |
| I-mm | >12.5 | >12.5 | >12.5 | <0.19 | <0.19 |
| I-nn | >12.5 | >12.5 | >12.5 | <0.19 | <0.19 |

TABLE L
Antifungal Activity

| | MIC2 (μg/mL) | | |
|---|---|---|---|
| | H. capsulatum | | |
| Compound | 05-959 | 05-1097 | 05-1159 |
| Ambruticin VS-4 | ≦0.15 | ≦0.15 | ≦0.15 |
| IV-a | ≦0.15 | ≦0.15 | ≦0.15 |

### EXAMPLE 26

The compounds of this invention were tested against two strains of Candida albicans, and one strain each of Candida krusei and Candida parapsilosis. The antifungal activity of the compounds tested were determined by microdilution methods according to the NCCLS reference method no. M27-A2, “Reference Method for Broth Dilution Antifungal Susceptibility Testing of Yeast Fungi; Approved Standard”. Results are presented in Tables M, N and T. MIC₀ and MIC indicates the minimal concentration observed to inhibit 95% and 50%, respectively, growth of the fungal species tested relative the untreated control. Where multiple assays were performed and a range of values was obtained, the minimum and maximum values are reported.

TABLE T
Antifungal Activity

| | MIC2 (μg/mL) | | |
|---|---|---|---|
| | | | C. albicans |
| Compound | C. albicans 05-1422 | C. albicans 05-1424 | 05-1426 |
| IV-a | ≦0.15 | >10 | >10 |

TABLE M
Antifungal Activity

| | MIC₀ (μg/mL) | | |
|---|---|---|---|
| | C. albicans | C. krusei | C. parapsilosis |
| Compound | 90028 | 6258 | 92019 |
| Amphotericin B | 1 | 1 | 1 |
| Itraconazole | 0.25 | 0.25 | 0.25 |
| IV-a | >32 | >32 | <0.0625 |

TABLE N
Antifungal Activity

MIC (μg/mL)

| Compound | C. albicans 24433 | C. albicans 90028 |
|---|---|---|
| Amphotericin B | 2-4 | 2-4 |
| Caspofungin | 2 | 2 |
| I-h | >32 | >32 |
| I-n | >32 | >32 |
| I-z | >32 | >32 |
| I-v | >32 | >32 |
| I-w | >32 | >32 |
| I-ff | 32 | 32 |
| I-hh | 32 | 32 |

### EXAMPLE 27

The compounds of this invention were tested against 3 different strains of Cryptococcus neoformans. The antifungal activity of the compounds tested were determined by microdilution methods according to the NCCLS reference method no. M38-A, “Reference Method for Broth Dilution Antifungal Susceptibility Testing of Filamentous Fungi; Approved Standard”. Results are presented in Table O. MIC indicates the minimal concentration observed to inhibit 50% growth of the fungal species tested relative to the untreated control. Where multiple assays were performed and a range of values was obtained, the minimum and maximum values are reported.

TABLE O
Antifungal Activity

| | MIC (μg/mL) | | |
|---|---|---|---|
| | C. neoformans | C. neoformans | C. neoformans |
| Compound | 97-14 | 11239 | 11240 |
| Amphotericin B | 0.5-2 | 0.5-1 | 0.5-1 |
| Itraconazole | 0.125 | 0.031 | 0.313 |
| Ambruticin S | 8 | 4 | 16 |
| 5-keto ambruticin S | 0.5 | 2 | 2 |
| I-a | 1 | 2 | 2 |
| I-b | 2 | 2 | 2 |
| I-c | 1 | 2 | 2 |
| I-h | 0.5 | 0.25 | 0.25 |
| I-n | 0.25 | 0.125-0.25 | 0.125-0.25 |
| I-z | 0.5-1 | 0.25-0.5 | 0.5 |
| I-v | 0.5 | 0.25 | 0.25-0.5 |
| I-t | 0.5 | 1 | 0.5 |
| I-u | 1 | 2 | 2 |
| I-w | 1 | 0.5 | 0.5-1 |
| I-y | 0.5 | 1 | 1 |
| I-ee | 2 | 2 | 2 |
| I-hh | 1 | 1 | 1 |
| I-ii | 0.5-1 | 0.25 | 0.25-0.5 |
| I-jj | 0.125 | 2 | 2 |
| I-kk | 0.25 | 0.125 | 0.0625 |
| I-ll | 0.5 | 1 | 0.5 |
| I-mm | 0.125 | 0.25 | 0.0625 |

### EXAMPLE 28

The compounds of this invention were tested against one strain of Fusarium solani and 3 different strains of Scedosporium apiospennum. The antifungal activity of the compounds tested were determined by microdilution methods according to the NCCLS reference method no. M38-A, “Reference Method for Broth Dilution Antifungal Susceptibility Testing of Filamentous Fungi; Approved Standard”. Results are presented in Table P. MIC indicates the minimal concentration observed to inhibit 50% growth of the fungal species tested relative to the untreated control. Where multiple assays were performed and a range of values was obtained, the minimum and maximum values are reported.

TABLE P
Antifungal Activity

| | MIC (μg/mL) | | | |
|---|---|---|---|---|
| | F. solani | S. apiospermum | S. apiospermum | S. apiospermum |
| Compound | 96-1 | 94-54 | 98-89 | 98-38 |
| Amphotericin B | 1-2 | >32 | 8 | 4 |
| Itraconazole | >4 | >2->4 | 1-2 | 1-4 |
| Ambruticin S | >32 | >32 | 4 | >32 |
| I-h | >16 | >16 | 0.125-0.25 | >16 |
| I-n | >16 | >16 | 0.125-0.25 | >16 |
| I-z | >16 | >16 | 0.25-0.5 | >16 |
| I-v | >16 | >16 | 0.25 | >16 |
| I-w | >16 | >16 | 2 | >16 |
| I-ff | 8-16 | 32 | 0.5 | 16 |
| I-hh | 16 | 32 | 1-2 | 32 |

### EXAMPLE 29

The compounds of this invention were tested against 15 different strains of 7 different dermatophyte species. The antifungal activity of the compounds tested were determined by microdilution methods according to the NCCLS reference method no. M38-A, “Reference Method for Broth Dilution Antifungal Susceptibility Testing of Filamentous Fungi; Approved Standard”. Results are presented in Table Q. MIC₀ indicates the minimal concentration observed to inhibit 95% growth of the fungal species tested relative to the untreated control.

TABLE Q
Antifungal Activity

| | MIC₀ (μg/mL) | | | | | |
|---|---|---|---|---|---|---|
| Compound | Tm | Mg | Ti | Ef | Tr | Mc |
| I-a | 5.65 | 2.51 | 2 | 4 | 2 | 1.41 |
| I-d | 8 | 2.51 | 2.51 | 4 | 3.17 | 2.82 |
| I-I | >8 | >8 | >8 | >8 | >8 | >8 |
| I-l | 5.66 | 0.79 | 0.79 | 2.82 | 1.58 | 1.41 |
| I-n | 8 | 1.99 | 1.25 | 2 | 2.51 | 2.82 |
| I-q | >8 | >8 | 12.69 | >8 | >8 | >8 |
| I-t | 11.31 | 2 | 1.25 | 1.41 | 1.58 | 2.82 |
| I-u | 11.31 | 4 | 3.17 | 2.82 | 3.17 | 5.65 |
| I-v | 4 | 2 | 0.63/4 | 0.5/2 | 5.03 | 2 |
| I-y | 4.18 | 2.51 | 0.39/2 | 0.71/2 | 3.17 | 4 |
| I-z | 4 | 1.58 | 0.79 | 4 | 1.25 | 2 |
| I-hh | 2.82 | 1.25 | 3.17 | 2.82 | 2.51 | 0.7 |
| I-ii | 1.41 | 1 | 1.58 | 1 | 1.58 | 0.5 |
| I-jj | >8 | 1.25 | 2.51 | 5.65 | 3.17 | 8 |
| I-mm | 2.82 | 0.79 | 0.79 | 1.41 | 1.25 | 0.7 |
| I-pp | 4 | 2.51 | 5.03 | 2.82 | ¼ | 1 |
| IV-a | 1 | 0.5 | 0.39 | 4 | 0.5 | 0.5 |
| IV-b | 4 | 1.25 | 2.51 | 1.41 | 2.51 | 1.41 |

Note:
Tm: Trichophyton mentagrophytes (2 strains);
Mg: Microsporum gypseum (3 strains);
Ti: Trichophyton interdigitale (3 strains);
Ef: Epidermophyton floccosum (2 strains);
Tr: Trichophyton rubrum (3 strains);
Mc: Microsporum canis (2 strains).

### EXAMPLE 30

The antifungal activities of compounds of this invention against two different Coccidioides species were determined by microdilution methods according to the NCCLS reference method no. M38-A, “Reference Method for Broth Dilution Antifungal Susceptibility Testing of Filamentous Fungi; Approved Standard”. Results are presented in Tables R and S. MIC indicates the minimal concentration observed to inhibit 80% growth of the fungal species tested relative to the untreated control. MIC2 indicates the minimal concentration observed to inhibit 50% growth of the fungal species tested relative to the untreated control. Where multiple assays were performed and a range of values was obtained, the minimum and maximum values are reported.

TABLE R
Antifungal Activity

| | MIC (μg/mL) | | | | | |
|---|---|---|---|---|---|---|
| | C. posadasii | | | C. immitis | | |
| | | | RMSCC | | | RMS |
| Compound | Silveria | C735 | 2127 | RS | S46 | 2281 |
| Amphotericin B | 1 | 1 | 1 | 1 | 1 | 1 |
| Fluconazole | 4 | 16 | 16 | 16 | 16 | 16 |
| Ambruticin S | 2 | 4 | 2 | 2 | 4 | 2 |
| I-v | 0.25 | 0.25 | 0.125 | 0.25 | 0.125 | 0.125 |
| I-ii | 0.5 | 0.5 | 0.25 | 0.25 | 0.25 | 0.125 |

TABLE S
Antifungal Activity

| | MIC2 (μg/mL) | | |
|---|---|---|---|
| | | | C. immitis |
| Compound | C. immitis 05-469 | C. immitis 05-955 | 05-1387 |
| Ambruticin VS-4 | ≦0.15 | ≦0.15 | ≦0.15 |
| IV-a | ≦0.15 | ≦0.15 | ≦0.15 |

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

What is claimed is:

1. A compound represented by formula I 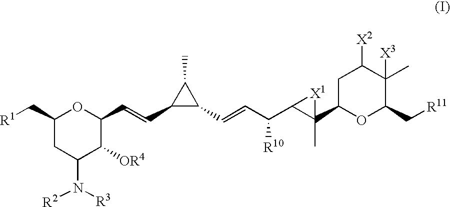
and the pharmaceutically acceptable salts, solvates, hydrates, and prodrug forms thereof, wherein
R¹⁰ and R¹¹ are independently H or CH₃;
X¹ is either a bond or O;
X² and X³ are each H or together are a bond;
R¹ is 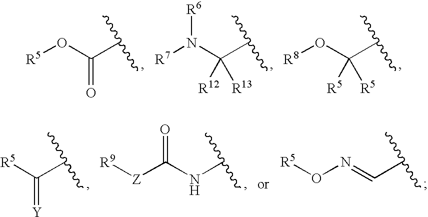
R² and R³ are independently H, C₁-C₅ alkyl, C₂-C₅ alkenyl, C₂-C₅ alkynyl, cycloalkyl, aryl(C₁-C₅ alkyl), aryl(C₂-C₅ alkenyl), aryl(C₂-C₅ alkynyl), cycloalkyl(C₁-C₅ alkyl), cycloalkyl(C₂-C₅ alkenyl), cycloalkyl(C₂-C₅ alkynyl), 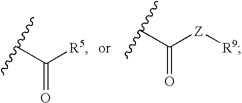
R⁴ is H, 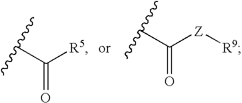
or R³ and R⁴ combine to form 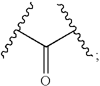
R⁵ is, independently for each occurrence thereof, H, C₁-C₅ alkyl, C₂-C₅ alkenyl, C₂-C₅ alkynyl, cycloalkyl, or aryl;
R⁶ and R⁷ are independently H, C₁-C₅ alkyl, C₂-C₅ alkenyl, C₂-C₅ alkynyl, cycloalkyl, or aryl; or R⁶ and R⁷ and the nitrogen to which they are commonly bonded combine to form an aziridinyl, azetidinyl, pyrrolidinyl, or piperidinyl ring;
R⁸ is R⁵ or 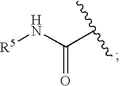
R⁹ is, independently for each occurrence thereof, H, C₁-C₅ alkyl, C₂-C₅ alkenyl, C₂-C₅ alkynyl, cycloalkyl, aryl(C₁-C₅ alkyl), aryl(C₂-C₅ alkenyl), aryl(C₂-C₅ alkynyl), cycloalkyl(C₁-C₅ alkyl), cycloalkyl(C₂-C₅ alkenyl), or cycloalkyl(C₂-C₅ alkynyl), provided that R⁹ is not H when Z is O;
R¹² and R¹³ together are 0, or R¹² is H and R¹³ is R⁵;
Y is O or N—OR⁵; and
Z is, independently for each occurrence thereof, O or NH;
with the proviso that when R¹ is 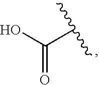
R¹⁰ and R¹¹ are both CH₃, X¹ is a bond, and R² is H or CH₃, then R³ is other than H or CH₃.

2. The compound according to claim 1, having a structure represented by formula II:

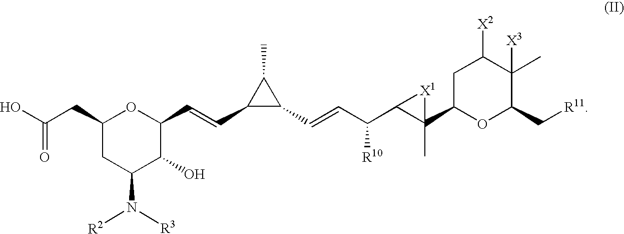

3. The compound according to claim 1, having a structure represented by formula III:

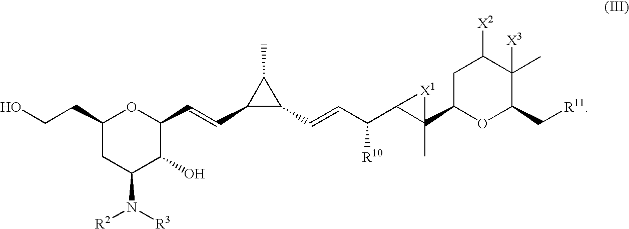

4. The compound according to claim 1, having a structure represented by formula IV:

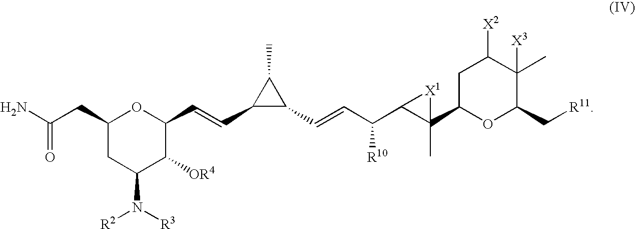

5. The compound according to claim 1, having a structure represented by formula I-A:

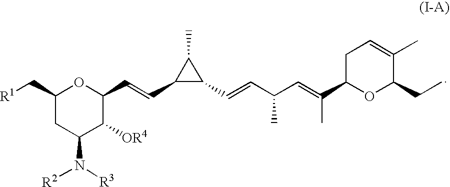

6. The compound according to claim 1, having a structure represented by formula II-A:

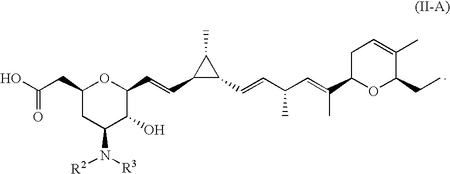

7. The compound according to claim 1, having a structure represented by formula III-A:

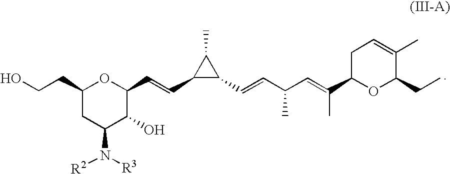

8. The compound according to claim 1, having a structure represented by formula IV-A:

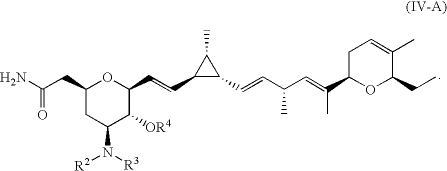

9. The compound according to claim 1, having a structure represented by formula I-B:

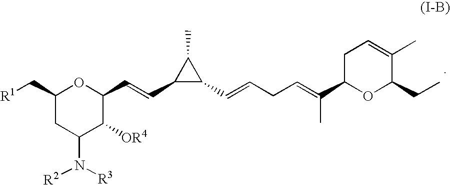

10. The compound according to claim 1, having a structure represented by formula II-B:

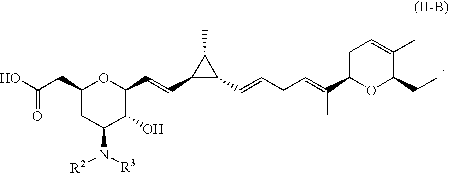

11. The compound according to claim 1, having a structure represented by formula III-B:

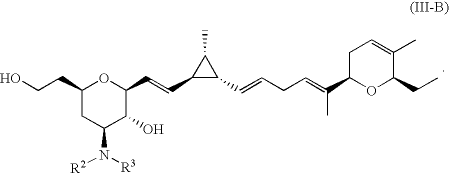

12. The compound according to claim 1, having a structure represented by formula IV-B:

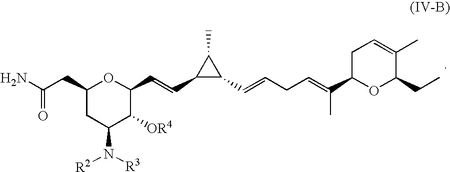

13. The compound according to claim 1, having a structure represented by formula I-C:

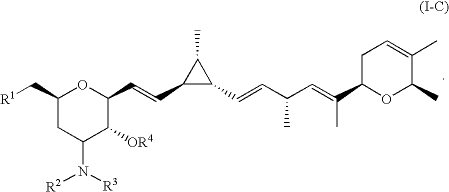

14. The compound according to claim 1, having a structure represented by formula II-C:

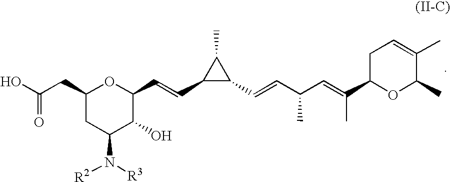

15. The compound according to claim 1, having a structure represented by formula III-C:

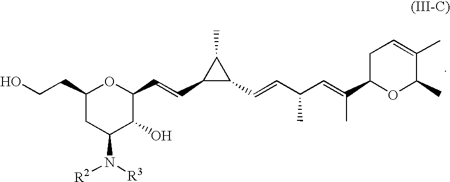

16. The compound according to claim 1, having a structure represented by formula IV-C:

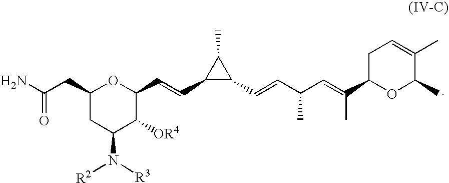

17. The compound according to claim 1, having a structure represented by formula I-D:

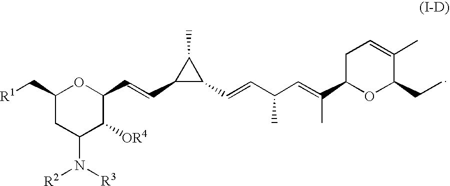

18. The compound according to claim 1, having a structure represented by formula II-D:

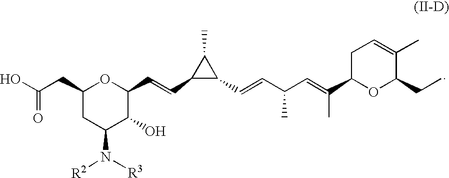

19. The compound according to claim 1, having a structure represented by formula III-D:

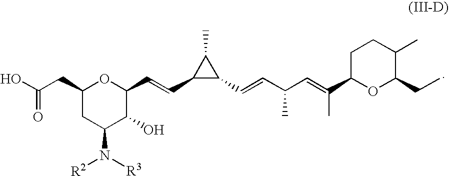

20. The compound according to claim 1, having a structure represented by formula IV-D:

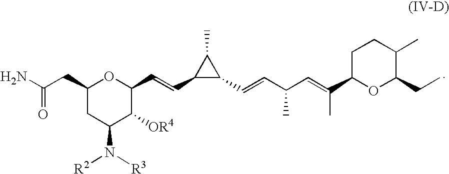

21. The compound according to claim 1, having a structure represented by formula I-E:

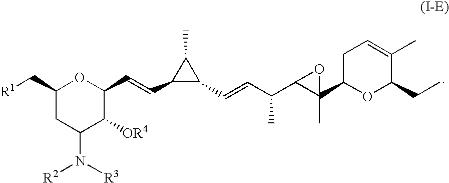

22. The compound according to claim 1, having a structure represented by formula II-E:

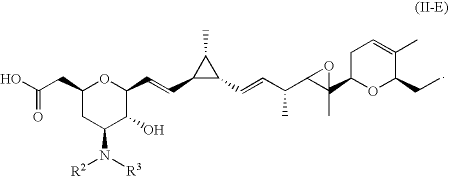

23. The compound according to claim 1, having a structure represented by formula III-E:

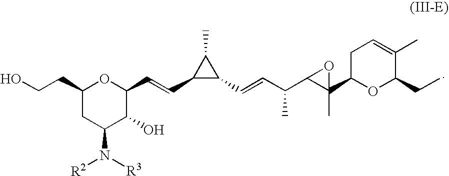

24. The compound according to claim 1, having a structure represented by formula IV-E:

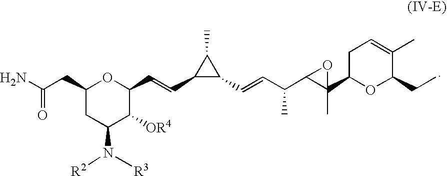

25. The compound according to claims 1-24, wherein R² is H, CH₃, aryl(CH₂), cycloalkyl(CH₂), or cycloalkyl; and R³ is C₂-C₅ alkyl, aryl(CH₂), cycloalkyl(CH₂), or cycloalkyl.

26. The compound according to claims 1-24, wherein R² is H, CH₃, CH₃CH₂, HOCH₂CH₂,

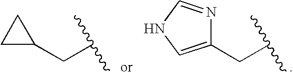

27. The compound according to claims 1-24, wherein R³ is CH₃CH₂, CH₂CH₂OH, (CH₃)₂CH, CH₃CH₂CH₂, CH₃CH₂CH₂CH₂,

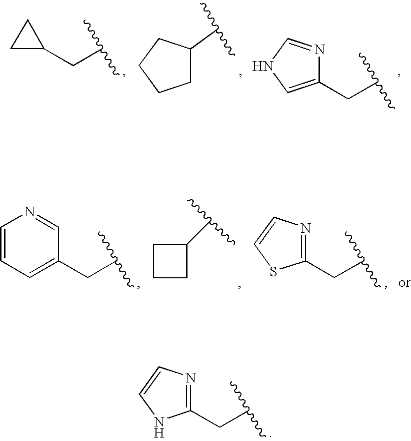

28. The compound according to claim 2, 6, 10, 14, 18 or 22, wherein R² and R³ are according to the combinations set forth in the following table:

| R² | R³ |
|---|---|
| H | 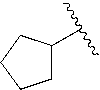 |
| 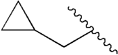 | 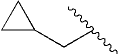 |
| 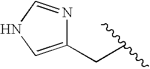 | 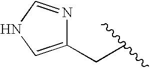 |
| CH₃CH₂ | CH₃CH₂ |
| HOCH₂CH₂ | HOCH₂CH₂ |
| H | 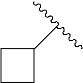 |
| H | (CH₃)₂CH |
| H | HOCH₂CH₂ |
| H | CH₃CH₂ |

29. The compound according to claim 2, 6, 10, 14, 18 or 22, wherein R² is H, and R² is selected from the group consisting of CH₃CH₂, HOCH₂CH₂, (CH₃)₂CH, COCF₃, CH₂CHF₂,

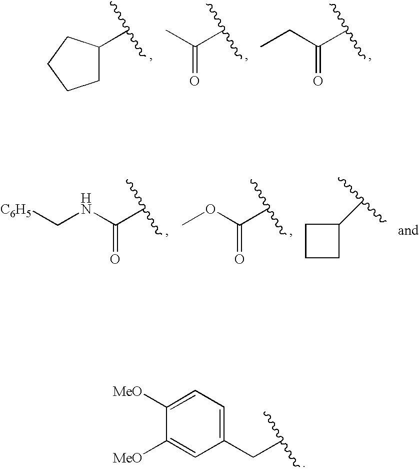

30. The compound according to claim 2, 6, 10, 14, 18 or 22, wherein R² is CH₃ or CH₃CH₂, and R⁵³ is selected from the group consisting of CH₃CH₂, HOCH₂CH₂, (CH₃)₂CH,

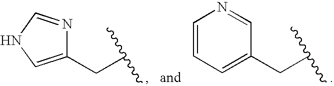

31. The compound according to claim 3, 7, 11, 15, 19 or 23, wherein R² is H, and R³ is selected from the group consisting of CH₃, COCF₃, and CH₂CHF₂.

32. The compound according to claim 3, 7, 11, 15, 19 or 23, wherein R² is CH₃, and R³ is selected from the group consisting of CH₃, HOCH₂CH₂, and (CH₃)₂CH.

33. The compound according to claim 1, 5, 9, 13, 17 or 21, wherein R¹ is 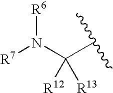
and R¹² and R¹³ together are O.

34. The compound according to claim 33, wherein R¹ is selected from the group consisting of

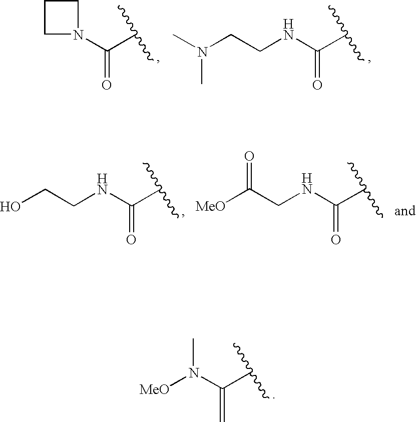

35. The compound according to claim 1, 5, 9, 13, 17 or 21, wherein R¹ is 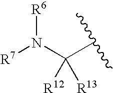
and R¹² is H and R¹³ is H.

36. The compound according to claim 35, wherein R¹ is selected from the group consisting of

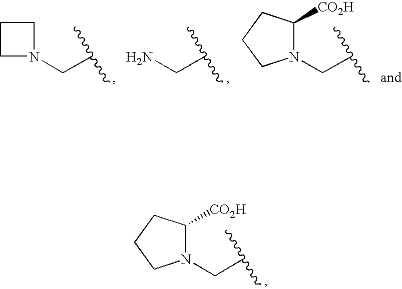

37. The compound according to claim 1, 5, 9, 13, 17 or 21, wherein R¹ is

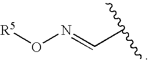

38. The compound according to claim 37, wherein R¹ is selected from the group consisting of

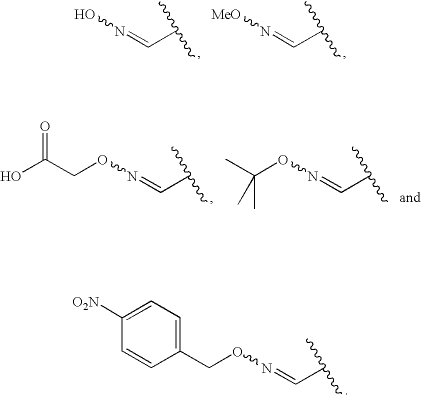

39. The compound according to claim 1, 5, 9, 13, 17 or 21, wherein R¹ is selected from the group consisting of

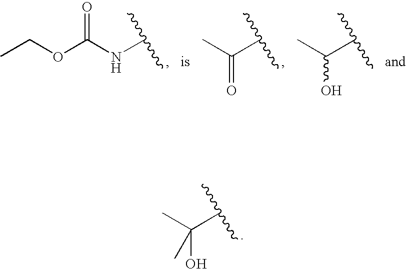

40. A method for treating a fungal infection, comprising administering to a subject suffering from such infection a therapeutically effective amount of a compound according to claim 1.

41. The method of claim 40, wherein said fungal infection is caused by a fungal species of a genus selected from the group consisting of Aspergillus, Blastomyces, Candida, Coccidiodes, Crytococcus, Epidermophyton, Fusarium, Hansenula, Histoplamsma, Microsporium, Mucor, Pseudallescheria, Rhizopus, Scedosporium, and Trichophyton.

42. The use of a compound according to claim 1 for the preparation of a medicament for treating a fungal infection.

43. A pharmaceutical formulation comprising a compound according to claim 1 and an excipient.

44. An isolated or recombinant cell comprising the genes of the ambruticin biosynthetic gene cluster and capable of producing one or more ambruticins or ambruticin analogs, wherein the activity the ambP, ambO, ambS, or ambM gene product is reduced or disrupted.

45. The cell of claim 44, wherein the activity the ambM gene product is reduced or disrupted.

46. The cell of claim 44, wherein the activity the ambP or ambO gene product is reduced or disrupted.

47. The cell of claim 44, wherein the activity the ambS gene product is reduced or disrupted.

48. An isolated or recombinant cell comprising the genes of the ambruticin biosynthetic gene cluster and capable of producing one or more ambruticins or ambruticin analogs, wherein the malonate specific AT domain from module 7 is replaced or engineered into a loading domain.

49. The cell of claims 44-48, wherein the cell is a Sorangium or Myxococcus cell.

50. A method of producing one or more ambruticins or ambruticin analogs comprising culturing the cell of claims 44-49.

51. An isolated or purified compound represented by the formula (II-D): 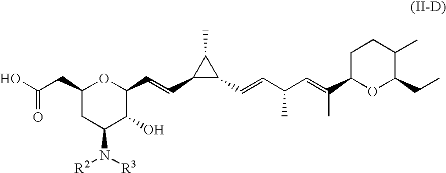
wherein R² and R³ are, independently for each occurrence thereof, H or CH₃.


SEQUENCE LISTING (16 sequences)

SEQ ID NO: 1 - DNA, 21 bp, Artificial Sequence
  chemically synthesized
tgatacaacgacgcttacacg

SEQ ID NO: 2 - DNA, 21 bp, Artificial Sequence
  chemically synthesized
ctagcggaacgacatggtgaa

SEQ ID NO: 3 - DNA, 21 bp, Artificial Sequence
  chemically synthesized
taggccaggttgagccatgag

SEQ ID NO: 4 - DNA, 21 bp, Artificial Sequence
  chemically synthesized
ctattgctctctggccaggag

SEQ ID NO: 5 - DNA, 21 bp, Artificial Sequence
  chemically synthesized
tgagcggtcggcgccagctgg

SEQ ID NO: 6 - DNA, 21 bp, Artificial Sequence
  chemically synthesized
tcacgtgaagcgcgccgcgtc

SEQ ID NO: 7 - DNA, 21 bp, Artificial Sequence
  chemically synthesized
tgacacccggtactcctcagc

SEQ ID NO: 8 - DNA, 21 bp, Artificial Sequence
  chemically synthesized
tcagcgcttgtccgccagacg

SEQ ID NO: 9 - DNA, 32 bp, Artificial Sequence
  chemically synthesized
ttttaattaagaggagcatatggatccgcagc

SEQ ID NO: 10 - DNA, 39 bp, Artificial Sequence
  chemically synthesized
gcccgcggcggttccggggcctcctcggacaccacatgc

SEQ ID NO: 11 - DNA, 40 bp, Artificial Sequence
  chemically synthesized
gccatgtggtgctcgaggaggccccggaaccgccgcgggc

SEQ ID NO: 12 - DNA, 28 bp, Artificial Sequence
  chemically synthesized
tttctagacctagggccattgagcgccg

SEQ ID NO: 13 - DNA, 26 bp, Artificial Sequence
  chemically synthesized
aatggccctaggcagaccgtcgtcag

SEQ ID NO: 14 - DNA, 40 bp, Artificial Sequence
  chemically synthesized
tagcgctggcgctggaatgcgtaggtcggcagctccaccc

SEQ ID NO: 15 - DNA, 40 bp, Artificial Sequence
  chemically synthesized
gggtggagctgccgacctacgcattccagcgccagcgcta

SEQ ID NO: 16 - DNA, 30 bp, Artificial Sequence
  chemically synthesized
tttctagagatctagacgagcgcatcgatg